SEQUENCE LISTING (37 sequences)

SEQ ID NO: 1 - RNA, 19 nt, Artificial Sequence
  Synthetic siRNA against AXL receptor
acagcgagauuuaugacua

SEQ ID NO: 2 - RNA, 19 nt, Artificial Sequence
  Synthetic siRNA against AXL receptor
gguaccggcuggcguauca

SEQ ID NO: 3 - RNA, 19 nt, Artificial Sequence
  Synthetic siRNA against AXL receptor
gacgaaauccucuauguca

SEQ ID NO: 4 - RNA, 19 nt, Artificial Sequence
  Synthetic siRNA against AXL receptor
gaaggagacccguuaugga

SEQ ID NO: 5 - protein, 890 aa, Homo sapiens
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 6 - DNA, 4001 bp, Homo sapiens
agtggaaggagcgcggtggcgcgggagcggccccggggaccccgcgctgctgacggcggcgaccgcggccggaggcgggcgcgggtctcggaggcggtcgcctcagcaccgccccacgggcggccccagcccctcccgcagccctcctccctcccgctcccttcccgccgcctcctccccgccctcctccctcctcgctcgcgggccgggcccggcatggtgcggcgtcgccgccgatggcgctgaggcggagcatggggcggccggggctcccgccgctgccgctgccgccgccaccgcggctcgggctgctgctggcggctctggcttctctgctgctcccggagtccgccgccgcaggtctgaagctcatgggagccccggtgaagctgacagtgtctcaggggcagccggtgaagctcaactgcagtgtggaggggatggaggagcctgacatccagtgggtgaaggatggggctgtggtccagaacttggaccagttgtacatcccagtcagcgagcagcactggatcggcttcctcagcctgaagtcagtggagcgctctgacgccggccggtactggtgccaggtggaggatgggggtgaaaccgagatctcccagccagtgtggctcacggtagaaggtgtgccatttttcacagtggagccaaaagatctggcagtgccacccaatgcccctttccaactgtcttgtgaggctgtgggtccccctgaacctgttaccattgtctggtggagaggaactacgaagatcgggggacccgctccctctccatctgttttaaatgtaacaggggtgacccagagcaccatgttttcctgtgaagctcacaacctaaaaggcctggcctcttctcgcacagccactgttcaccttcaagcactgcctgcagcccccttcaacatcaccgtgacaaagctttccagcagcaacgctagtgtggcctggatgccaggtgctgatggccgagctctgctacagtcctgtacagttcaggtgacacaggccccaggaggctgggaagtcctggctgttgtggtccctgtgcccccctttacctgcctgctccgggacctggtgcctgccaccaactacagcctcagggtgcgctgtgccaatgccttggggccctctccctatgctgactgggtgccctttcagaccaagggtctagccccagccagcgctccccaaaacctccatgccatccgcacagattcaggcctcatcttggagtgggaagaagtgatccccgaggcccctttggaaggccccctgggaccctacaaactgtcctgggttcaagacaatggaacccaggatgagctgacagtggaggggaccagggccaatttgacaggctgggatccccaaaaggacctgatcgtacgtgtgtgcgtctccaatgcagttggctgtggaccctggagtcagccactggtggtctcttctcatgaccgtgcaggccagcagggccctcctcacagccgcacatcctgggtacctgtggtccttggtgtgctaacggccctggtgacggctgctgccctggccctcatcctgcttcgaaagagacggaaagagacgcggtttgggcaagcctttgacagtgtcatggcccggggagagccagccgttcacttccgggcagcccggtccttcaatcgagaaaggcccgagcgcatcgaggccacattggacagcttgggcatcagcgatgaactaaaggaaaaactggaggatgtgctcatcccagagcagcagttcaccctgggccggatgttgggcaaaggagagtttggttcagtgcgggaggcccagctgaagcaagaggatggctcctttgtgaaagtggctgtgaagatgctgaaagctgacatcattgcctcaagcgacattgaagagttcctcagggaagcagcttgcatgaaggagtttgaccatccacacgtggccaaacttgttggggtaagcctccggagcagggctaaaggccgtctccccatccccatggtcatcttgcccttcatgaagcatggggacctgcatgccttcctgctcgcctcccggattggggagaacccctttaacctacccctccagaccctgatccggttcatggtggacattgcctgcggcatggagtacctgagctctcggaacttcatccaccgagacctggctgctcggaattgcatgctggcagaggacatgacagtgtgtgtggctgacttcggactctcccggaagatctacagtggggactactatcgtcaaggctgtgcctccaaactgcctgtcaagtggctggccctggagagcctggccgacaacctgtatactgtgcagagtgacgtgtgggcgttcggggtgaccatgtgggagatcatgacacgtgggcagacgccatatgctggcatcgaaaacgctgagatttacaactacctcattggcgggaaccgcctgaaacagcctccggagtgtatggaggacgtgtatgatctcatgtaccagtgctggagtgctgaccccaagcagcgcccgagctttacttgtctgcgaatggaactggagaacatcttgggccagctgtctgtgctatctgccagccaggaccccttatacatcaacatcgagagagctgaggagcccactgcgggaggcagcctggagctacctggcagggatcagccctacagtggggctggggatggcagtggcatgggggcagtgggtggcactcccagtgactgtcggtacatactcacccccggagggctggctgagcagccagggcaggcagagcaccagccagagagtcccctcaatgagacacagaggcttttgctgctgcagcaagggctactgccacacagtagctgttagcccacaggcagagggcatcggggccatttggccggctctggtggccactgagctggctgactaagccccgtctgaccccagcccagacagcaaggtgtggaggctcctgtggtagtcctcccaagctgtgctgggaagcccggactgaccaaatcacccaatcccagttcttcctgcaaccactctgtggccagcctggcatcagtttaggccttggcttgatggaagtgggccagtcctggttgtctgaacccaggcagctggcaggagtggggtggttatgtttccatggttaccatgggtgtggatggcagtgtggggagggcaggtccagctctgtgggccctaccctcctgctgagctgcccctgctgcttaagtgcatgcattgagctgcctccagcctggtggcccagctattaccacacttggggtttaaatatccaggtgtgcccctccaagtcacaaagagatgtccttgtaatattcccttttaggtgagggttggtaaggggttggtatctcaggtctgaatcttcaccatctttctgattccgcaccctgcctacgccaggagaagttgaggggagcatgcttccctgcagctgaccgggtcacacaaaggcatgctggagtacccagcctatcaggtgcccctcttccaaaggcagcgtgccgagccagcaagaggaaggggtgctgtgaggcttgcccaggagcaagtgaggccggagaggagttcaggaacccttctccatacccacaatctgagcacgctaccaaatctcaaaatatcctaagactaacaaaggcagctgtgtctgagcccaacccttctaaacggtgacctttagtgccaacttcccctctaactggacagcctcttctgtcccaagtctccagagagaaatcaggcctgatgagggggaattcctggaacctggaccccagccttggtgggggagcctctggaatgcatggggcgggtcctagctgttagggacatttccaagctgttagttgctgtttaaaatagaaataaaattgaagactaaagacctaaaaaaaaaaaaaaaaaaaaaaaaaaaaaaaa

SEQ ID NO: 7 - protein, 885 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 8 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 9 - DNA, 4743 bp, Homo sapiens
gggaaggaggcaggggtgctgagaaggcggctgctgggcagagccggtggcaagggcctcccctgccgctgtgccaggcaggcagtgccaaatccggggagcctggagctggggggagggccggggacagcccggccctgccccctcccccgctgggagcccaacaacttctgaggaaagtttggcacccatggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagggcaagcacagccagtccaccagctggtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctgagacaaccctccacctggtactccctctcaggatccaagctaagcactgccactggggaaaactccaccttcccactttcccaccccacgccttatccccacttgcagccctgtcttcctacctatcccacctccatcccagacaggtccctccccttctctgtgcagtagcatcaccttgaaagcagtagcatcaccatctgtaaaaggaaggggttggattgcaatatctgaagccctcccaggtgttaacattccaagactctagagtccaaggtttaaagagtctagattcaaaggttctaggtttcaaagatgctgtgagtctttggttctaaggacctgaaattccaaagtctctaattctattaaagtgctaaggttctaaggcctactttttttttttttttttttttttttttttttgcgatagagtctcactgtgtcacccaggctggagtgcagtggtgcaatctcgcctcactgcaaccttcacctaccgagttcaagtgattttcctgccttggcctcccaagtagctgggattacaggtgtgtgccaccacacccggctaatttttatatttttagtagagacagggtttcaccatgttggccaggctggtctaaaactcctgacctcaagtgatctgcccacctcagcctcccaaagtgctgagattacaggcatgagccactgcactcaaccttaagacctactgttctaaagctctgacattatgtggttttagattttctggttctaacatttttgataaagcctcaaggttttaggttctaaagttctaagattctgattttaggagctaaggctctatgagtctagatgtttattcttctagagttcagagtccttaaaatgtaagattatagattctaaagattctatagttctagacatggaggttctaaggcctaggattctaaaatgtgatgttctaaggctctgagagtctagattctctggctgtaaggctctagatcataaggcttcaaaatgttatcttctcaagttctaagattctaatgatgatcaattatagtttctgaggctttatgataatagattctcttgtataagatcctagatcctaagggtcgaaagctctagaatctgcaattcaaaagttccaagagtctaaagatggagtttctaaggtccggtgttctaagatgtgatattctaagacttactctaagatcttagattctctgtgtctaagattctagatcagatgctccaagattctagatgattaaataagattctaacggtctgttctgtttcaaggcactctagattccattggtccaagattccggatcctaagcatctaagttataagactctcacactcagttgtgactaactagacaccaaagttctaataatttctaatgttggacacctttaggttctttgctgcattctgcctctctaggaccatggttaagagtccaagaatccacatttctaaaatcttatagttctaggcactgtagttctaagactcaaatgttctaagtttctaagattctaaaggtccacaggtctagactattaggtgcaatttcaaggttctaaccctatactgtagtattctttggggtgcccctctccttcttagctatcattgcttcctcctccccaactgtgggggtgtgcccccttcaagcctgtgcaatgcattagggatgcctcctttcccgcaggggatggacgatctcccacctttcgggccatgttgcccccgtgagccaatccctcaccttctgagtacagagtgtggactctggtgcctccagaggggctcaggtcacataaaactttgtatatcaacgaaaaaaa

SEQ ID NO: 10 - DNA, 4716 bp, Homo sapiens
gggaaggaggcaggggtgctgagaaggcggctgctgggcagagccggtggcaagggcctcccctgccgctgtgccaggcaggcagtgccaaatccggggagcctggagctggggggagggccggggacagcccggccctgccccctcccccgctgggagcccaacaacttctgaggaaagtttggcacccatggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctgagacaaccctccacctggtactccctctcaggatccaagctaagcactgccactggggaaaactccaccttcccactttcccaccccacgccttatccccacttgcagccctgtcttcctacctatcccacctccatcccagacaggtccctccccttctctgtgcagtagcatcaccttgaaagcagtagcatcaccatctgtaaaaggaaggggttggattgcaatatctgaagccctcccaggtgttaacattccaagactctagagtccaaggtttaaagagtctagattcaaaggttctaggtttcaaagatgctgtgagtctttggttctaaggacctgaaattccaaagtctctaattctattaaagtgctaaggttctaaggcctactttttttttttttttttttttttttttttttgcgatagagtctcactgtgtcacccaggctggagtgcagtggtgcaatctcgcctcactgcaaccttcacctaccgagttcaagtgattttcctgccttggcctcccaagtagctgggattacaggtgtgtgccaccacacccggctaatttttatatttttagtagagacagggtttcaccatgttggccaggctggtctaaaactcctgacctcaagtgatctgcccacctcagcctcccaaagtgctgagattacaggcatgagccactgcactcaaccttaagacctactgttctaaagctctgacattatgtggttttagattttctggttctaacatttttgataaagcctcaaggttttaggttctaaagttctaagattctgattttaggagctaaggctctatgagtctagatgtttattcttctagagttcagagtccttaaaatgtaagattatagattctaaagattctatagttctagacatggaggttctaaggcctaggattctaaaatgtgatgttctaaggctctgagagtctagattctctggctgtaaggctctagatcataaggcttcaaaatgttatcttctcaagttctaagattctaatgatgatcaattatagtttctgaggctttatgataatagattctcttgtataagatcctagatcctaagggtcgaaagctctagaatctgcaattcaaaagttccaagagtctaaagatggagtttctaaggtccggtgttctaagatgtgatattctaagacttactctaagatcttagattctctgtgtctaagattctagatcagatgctccaagattctagatgattaaataagattctaacggtctgttctgtttcaaggcactctagattccattggtccaagattccggatcctaagcatctaagttataagactctcacactcagttgtgactaactagacaccaaagttctaataatttctaatgttggacacctttaggttctttgctgcattctgcctctctaggaccatggttaagagtccaagaatccacatttctaaaatcttatagttctaggcactgtagttctaagactcaaatgttctaagtttctaagattctaaaggtccacaggtctagactattaggtgcaatttcaaggttctaaccctatactgtagtattctttggggtgcccctctccttcttagctatcattgcttcctcctccccaactgtgggggtgtgcccccttcaagcctgtgcaatgcattagggatgcctcctttcccgcaggggatggacgatctcccacctttcgggccatgttgcccccgtgagccaatccctcaccttctgagtacagagtgtggactctggtgcctccagaggggctcaggtcacataaaactttgtatatcaacgaaaaaaa

SEQ ID NO: 11 - protein, 999 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 12 - DNA, 3632 bp, Homo sapiens
actcactgcccgggccgcccggacagggagcttcgctggcgcgcttggccggcgacaggacaggttcgggacgtccatctgtccatccgtccggagagaaattacagatccgcagccccgggatggggccggccccgctgccgctgctgctgggcctcttcctccccgcgctctggcgtagagctatcactgaggcaagggaagaagccaagccttacccgctattcccgggaccttttccagggagcctgcaaactgaccacacaccgctgttatcccttcctcacgccagtgggtaccagcctgccttgatgttttcaccaacccagcctggaagaccacatacaggaaacgtagccattccccaggtgacctctgtcgaatcaaagcccctaccgcctcttgccttcaaacacacagttggacacataatactttctgaacataaaggtgtcaaatttaattgctcaatcagtgtacctaatatataccaggacaccacaatttcttggtggaaagatgggaaggaattgcttggggcacatcatgcaattacacagttttatccagatgatgaagttacagcaataatcgcttccttcagcataaccagtgtgcagcgttcagacaatgggtcgtatatctgtaagatgaaaataaacaatgaagagatcgtgtctgatcccatctacatcgaagtacaaggacttcctcactttactaagcagcctgagagcatgaatgtcaccagaaacacagccttcaacctcacctgtcaggctgtgggcccgcctgagcccgtcaacattttctgggttcaaaacagtagccgtgttaacgaacagcctgaaaaatccccctccgtgctaactgttccaggcctgacggagatggcggtcttcagttgtgaggcccacaatgacaaagggctgaccgtgtccaagggagtgcagatcaacatcaaagcaattccctccccaccaactgaagtcagcatccgtaacagcactgcacacagcattctgatctcctgggttcctggttttgatggatactccccgttcaggaattgcagcattcaggtcaaggaagctgatccgctgagtaatggctcagtcatgatttttaacacctctgccttaccacatctgtaccaaatcaagcagctgcaagccctggctaattacagcattggtgtttcctgcatgaatgaaataggctggtctgcagtgagcccttggattctagccagcacgactgaaggagccccatcagtagcacctttaaatgtcactgtgtttctgaatgaatctagtgataatgtggacatcagatggatgaagcctccgactaagcagcaggatggagaactggtgggctaccggatatcccacgtgtggcagagtgcagggatttccaaagagctcttggaggaagttggccagaatggcagccgagctcggatctctgttcaagtccacaatgctacgtgcacagtgaggattgcagccgtcaccagagggggagttgggcccttcagtgatccagtgaaaatatttatccctgcacacggttgggtagattatgccccctcttcaactccggcgcctggcaacgcagatcctgtgctcatcatctttggctgcttttgtggatttattttgattgggttgattttatacatctccttggccatcagaaaaagagtccaggagacaaagtttgggaatgcattcacagaggaggattctgaattagtggtgaattatatagcaaagaaatccttctgtcggcgagccattgaacttaccttacatagcttgggagtcagtgaggaactacaaaataaactagaagatgttgtgattgacaggaatcttctaattcttggaaaaattctgggtgaaggagagtttgggtctgtaatggaaggaaatcttaagcaggaagatgggacctctctgaaagtggcagtgaagaccatgaagttggacaactcttcacagcgggagatcgaggagtttctcagtgaggcagcgtgcatgaaagacttcagccacccaaatgtcattcgacttctaggtgtgtgtatagaaatgagctctcaaggcatcccaaagcccatggtaattttacccttcatgaaatacggggacctgcatacttacttactttattcccgattggagacaggaccaaagcatattcctctgcagacactattgaagttcatggtggatattgccctgggaatggagtatctgagcaacaggaattttcttcatcgagatttagctgctcgaaactgcatgttgcgagatgacatgactgtctgtgttgcggacttcggcctctctaagaagatttacagtggcgattattaccgccaaggccgcattgctaagatgcctgttaaatggatcgccatagaaagtcttgcagaccgagtctacacaagtaaaagtgatgtgtgggcatttggcgtgaccatgtgggaaatagctacgcggggaatgactccctatcctggggtccagaaccatgagatgtatgactatcttctccatggccacaggttgaagcagcccgaagactgcctggatgaactgtatgaaataatgtactcttgctggagaaccgatcccttagaccgccccaccttttcagtattgaggctgcagctagaaaaactcttagaaagtttgcctgacgttcggaaccaagcagacgttatttacgtcaatacacagttgctggagagctctgagggcctggcccagggctccacccttgctccactggacttgaacatcgaccctgactctataattgcctcctgcactccccgcgctgccatcagtgtggtcacagcagaagttcatgacagcaaacctcatgaaggacggtacatcctgaatgggggcagtgaggaatgggaagatctgacttctgccccctctgctgcagtcacagctgaaaagaacagtgttttaccgggggagagacttgttaggaatggggtctcctggtcccattcgagcatgctgcccttgggaagctcattgcccgatgaacttttgtttgctgacgactcctcagaaggctcagaagtcctgatgtgaggagaggtgcggggagacattccaaaaatcaagccaattcttctgctgtaggagaatccaattgtacctgatgtttttggtatttgtcttccttaccaagtgaactccatggccccaaagcaccagatgaatgttgttaagtaagctgtcattaaaaatacataatatatatttatttaaagagaaaaaatatgtgtatatcatggaaaaagacaaggatattttaataaaacattacttatttcatttcacttatcttgcatatcttaaaattaagcttcagctgctccttgatattaacatttgtacagagttgaagttgttttttcaagttcttttctttttcatgactattaaatgtaaaaatatttgtaaaatgaaatgccatatttgacttggcttctggtcttgatgtatttgataagaatgattcattcaatgtttaaagttgtataactgattaattttctgatatggcttcctaataaaatatgaataaggaagaaaaaa

SEQ ID NO: 13 - protein, 678 aa, Homo sapiens
MAPSLSPGPAALRRAPQLLLLLLAAECALAALLPAREATQFLRPRQRRAFQVFEEAKQGHLERECVEELCSREEAREVFENDPETDYFYPRYLDCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 14 - protein, 405 aa, Homo sapiens
MDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 15 - protein, 379 aa, Homo sapiens
MFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 16 - DNA, 2521 bp, Homo sapiens
ccgagcgcttgaggtgccgcagccgccgccgccgccgccgccgcgatgtgaccttcagggccgccaggacgggatgaccggagcctccgccccgcggcgcccgcggctcgcctcggcctcccgggcgctctgaccgcgcgtccccggcccgccatggccccttcgctctcgcccgggcccgccgccctgcgccgcgcgccgcagctgctgctgctgctgctggccgcggagtgcgcgcttgccgcgctgttgccggcgcgcgaggccacgcagttcctgcggcccaggcagcgccgcgcctttcaggtcttcgaggaggccaagcagggccacctggagagggagtgcgtggaggagctgtgcagccgcgaggaggcgcgggaggtgttcgagaacgaccccgagacggattatttttacccaagatacttagactgcatcaacaagtatgggtctccgtacaccaaaaactcaggcttcgccacctgcgtgcaaaacctgcctgaccagtgcacgcccaacccctgcgataggaaggggacccaagcctgccaggacctcatgggcaacttcttctgcctgtgtaaagctggctgggggggccggctctgcgacaaagatgtcaacgaatgcagccaggagaacgggggctgcctccagatctgccacaacaagccgggtagcttccactgttcctgccacagcggcttcgagctctcctctgatggcaggacctgccaagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctaggcccccacgggacgcggcaggcttctcagtctctgtccgagacagccgggaggagcctgggggctcctcaccacgtggggccatgctgagagctgggctttcctctgtgaccatcccggcctgtaacatatctgtaaatagtgagatggacttggggcctctgacgccgcgcactcagccgtgggcccgggcgcggggaggccggcgcagcgcagagcgggctcgaagaaaataattctctattatttttattaccaagcgcttctttctgactctaaaatatggaaaataaaatatttacagaaagctttgtaaaaaaaaaaaaaaaaaa

SEQ ID NO: 17 - DNA, 2188 bp, Homo sapiens
ttgattgaaaccagtaaatgcttctctttggggttggggttttagtttcaaatgcccccggggggttactttttacggccccgtgtcctgtagcaccgtcatttaaatggaacagcacagcgtgcaccgccgccccccacccctccaccaagcagggcccttcccagctctccacctgctgggctgaagtcagccttcccagccgggccttgatcagaagcgtgcaccaacaccccgggagctgcccggtcaggggaggagggcagggaaatggggccagggcgcgctggccccacagagtctggatgcgacctctgggtggtgccctggccagtccctgcagccgcctgccccagccccgtctgagatgccgctgtgctgcggttggccggtttttttttgcttgcagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctaggcccccacgggacgcggcaggcttctcagtctctgtccgagacagccgggaggagcctgggggctcctcaccacgtggggccatgctgagagctgggctttcctctgtgaccatcccggcctgtaacatatctgtaaatagtgagatggacttggggcctctgacgccgcgcactcagccgtgggcccgggcgcggggaggccggcgcagcgcagagcgggctcgaagaaaataattctctattatttttattaccaagcgcttctttctgactctaaaatatggaaaataaaatatttacagaaagctttgtaaaaaaaaaaaaaaaaaa

SEQ ID NO: 18 - DNA, 2523 bp, Homo sapiens
cacaccgacctgtcacaccggtgcctgtcacaccactgcctgtcacactgacttgtcaccggtgtctgtcacaccgacctgtcacactggtgcctgtcacactggtgcctgtcacaccgacctgtcacaccggtgcctgtcacaccgacctgtcacactgacctgtcacaccggtaggaatgcagtacccacatgtggacgtttctgggcagggcggctcttgtctttcctcttcagcctgggcctgtgcctgggggttgatgagagtgagcatttatttaaaaagcaaaaccacaggtggaaagagtcaccaggacagcttctcggagtcgcagacctgggatgcagccgtggggctcttgggtctgggctgcgacgttcagggcttccagccagccctcgccttgaggttctttgcctcgctgcctcatgtactcatgcagagggtgtcggacccctgcgagatgtccagctcaccctggctgcccacggtgggcagggcaggcctggctcagccccagcccctccatcttccaggggtgtcagctcacaccggctttggttctgtcccccttcgggcagcgtggagaaaccacagcccagaacagggaactttccaggacagccatcttcaaggcatccatatctatttcataatagtgtatactttttaatgattctctgtaatttttgtatgcttgaaatatttcataatttaaaaataaagggtcaagggaaatgagcagggaaggagatgacggggacccccgagaagccctgtgggaagcggctgctgcaagcccgcccttcacctgggagtcccagtggggcaggtgtgacagcctctggggtctcagcagctagaggcggggtggccactcccgaggcacaggagggacagtggacccgctgcgcggccggggcgtggggctcaggggagcaggagtgaaggccacatccccgaccggcgtggcccccgtccgtggcaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctaggcccccacgggacgcggcaggcttctcagtctctgtccgagacagccgggaggagcctgggggctcctcaccacgtggggccatgctgagagctgggctttcctctgtgaccatcccggcctgtaacatatctgtaaatagtgagatggacttggggcctctgacgccgcgcactcagccgtgggcccgggcgcggggaggccggcgcagcgcagagcgggctcgaagaaaataattctctattatttttattaccaagcgcttctttctgactctaaaatatggaaaataaaatatttacagaaagctttgtaaaaaaaaaaaaaaaaaa

SEQ ID NO: 19 - protein, 559 aa, Artificial Sequence
  Variant Gas6deltaGla protein
DQCTPNPCDKKGTHICQDLMGNFFCVCTDGWGGRLCDKDVNECVQKNGGCSQVCHNKPGSFQCACHSGFSLASDGQTCQDIDECTDSDTCGDARCKNLPGSYSCLCDEGYTYSSKEKTCQDVDECQQDRCEQTCVNSPGSYTCHCDGRGGLKLSPDMDTCEDILPCVPFSMAKSVKSLYLGRMFSGTPVIRLRFKRLQPTRLLAEFDFRTFDPEGVLFFAGGRSDSTWIVLGLRAGRLELQLRYNGVGRITSSGPTINHGMWQTISVEELERNLVIKVNKDAVMKIAVAGELFQLERGLYHLNLTVGGIPFKESELVQPINPRLDGCMRSWNWLNGEDSAIQETVKANTKMQCFSVTERGSFFPGNGFATYRLNYTRTSLDVGTETTWEVKVVARIRPATDTGVLLALVGDDDVVISVALVDYHSTKKLKKQLVVLAVEDVALALMEIKVCDSQEHTVTVSLREGEATLEVDGTKGQSEVSTAQLQERLDTLKTHLQGSVHTYVGGLPEVSVISAPVTAFYRGCMTLEVNGKILDLDTASYKHSDITSHSCPPVEHATP

SEQ ID NO: 20 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 21 - DNA, 4743 bp, Homo sapiens; identical to SEQ ID NO: 9

SEQ ID NO: 22 - protein, 364 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 23 - DNA, 1841 bp, Homo sapiens
attctcctgcctcagcctcccgagtagctgggactacaggcgccagtgaccacgcccggctaattttttgtatttttagtagagacggggtttcacccttttagccaggatggtctcgatctcctgacttcgtgatctgcccgccttggcctcccaaagtgctaggattacaggtttgagccaccgcgcccggccctgtttcctttttgtttgttcccctgataccctgtatcaggaccaggagtcagtttggcggttatgtgtggggaagaagctgggaagtcaggggctgtttctgtggacagctttccctgtcctttggaaggcacagagctctcagctgcagggaactaacagagctctgaagccgttatatgtggtcttctctcatttccagcagagcaggctcatatgaatcaaccaactgggtgaaaagataagttgcaatctgagatttaagacttgatcagataccatctggtggagggtaccaaccagcctgtctgctcattttccttcaggctgatcccataatgcatcctcaagtggtcatcttaagcctcatcctacatctggcagattctgtagctggttctgtaaaggttggtggagaggcaggtccatctgtcacactaccctgccactacagtggagctgtcacatccatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaatgacgactgttccaacgacaactgttccaacaacaatgagcattccaacgacaacgactgttctgacgacaatgactgtttcaacgacaacgagcgttccaacgacaacgagcattccaacaacaacaagtgttccagtgacaacaactgtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaagacccagtggtgctctttgagagtttacgcccatgagtgcagaagactgaacagacatcagcacatcagacgtcttttagaccccaagacaatttttctgtttcagtttcatctggcattccaacatgtcagtgatactgggtagagtaactctctcactccaaactgtgtatagtcaacctcatcattaatgtagtcctaattttttatgct

SEQ ID NO: 24 - DNA, 1493 bp, Homo sapiens
attctcctgcctcagcctcccgagtagctgggactacaggcgccagtgaccacgcccggctaattttttgtatttttagtagagacggggtttcacccttttagccaggatggtctcgatctcctgacttcgtgatctgcccgccttggcctcccaaagtgctaggattacaggctgatcccataatgcatcctcaagtggtcatcttaagcctcatcctacatctggcagattctgtagctggttctgtaaaggttggtggagaggcaggtccatctgtcacactaccctgccactacagtggagctgtcacatccatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaatgacgactgttccaacgacaactgttccaacaacaatgagcattccaacgacaacgactgttctgacgacaatgactgtttcaacgacaacgagcgttccaacgacaacgagcattccaacaacaacaagtgttccagtgacaacaactgtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaagacccagtggtgctctttgagagtttacgcccatgagtgcagaagactgaacagacatcagcacatcagacgtcttttagaccccaagacaatttttctgtttcagtttcatctggcattccaacatgtcagtgatactgggtagagtaactctctcactccaaactgtgtatagtcaacctcatcattaatgtagtcctaattttttatgct

SEQ ID NO: 25 - DNA, 1359 bp, Homo sapiens
gttacccagcattgtgagtgacagagcctggatctgaacgctgatcccataatgcatcctcaagtggtcatcttaagcctcatcctacatctggcagattctgtagctggttctgtaaaggttggtggagaggcaggtccatctgtcacactaccctgccactacagtggagctgtcacatccatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaatgacgactgttccaacgacaactgttccaacaacaatgagcattccaacgacaacgactgttctgacgacaatgactgtttcaacgacaacgagcgttccaacgacaacgagcattccaacaacaacaagtgttccagtgacaacaactgtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaagacccagtggtgctctttgagagtttacgcccatgagtgcagaagactgaacagacatcagcacatcagacgtcttttagaccccaagacaatttttctgtttcagtttcatctggcattccaacatgtcagtgatactgggtagagtaactctctcactccaaactgtgtatagtcaacctcatcattaatgtagtcctaattttttatgct

SEQ ID NO: 26 - protein, 142 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPGEWTFACHLYE

SEQ ID NO: 27 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 28 - DNA, 2448 bp, Homo sapiens
agaacacttacaggatgtgtgtagtgtggcatgacagagaactttggtttcctttaatgtgactgtagacctggcagtgttactataagaatcactggcaatcagacacccgggtgtgctgagctagcactcagtgggggcggctactgctcatgtgattgtggagtagacagttggaagaagtacccagtccatttggagagttaaaactgtgcctaacagaggtgtcctctgacttttcttctgcaagctccatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactcggcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatagatccaaccaccttatttttgagcttggtgttttgtctttttcagaaactatgagctgtgtcacctgactggttttggaggttctgtccactgctatggagcagagttttcccattttcagaagataatgactcacatgggaattgaactgggacctgcactgaacttaaacaggcatgtcattgcctctgtatttaagccaacagagttacccaacccagagactgttaatcatggatgttagagctcaaacgggcttttatatacactaggaattcttgacgtggggtctctggagctccaggaaattcgggcacatcatatgtccatgaaacttcagataaactagggaaaactgggtgctgaggtgaaagcataacttttttggcacagaaagtctaaaggggccactgattttcaaagagatctgtgatccctttttgttttttgtttttgagatggagtcttgctctgttgcccaggctggagtgcaatggcacaatctcggctcactgcaagctccgcctcctgggttcaagcgattctcctgcctcagcctcctgagtggctgggattacaggcatgcaccaccatgcccagctaatttgttgtatttttagtagagacagggtttcaccatgttggccagtgtggtctcaaactcctgacctcatgatttgcctgcctcggcctcccaaagcactgggattacaggcgtgagccaccacatccagccagtgatccttaaaagattaagagatgactggaccaggtctaccttgatcttgaagattcccttggaatgttgagatttaggcttatttgagcactgcctgcccaactgtcagtgccagtgcatagcccttcttttgtctcccttatgaagactgccctgcagggctgagatgtggcaggagctcccagggaaaaacgaagtgcatttgattggtgtgtattggccaagttttgcttgttgtgtgcttgaaagaaaatatctctgaccaacttctgtattcgtggaccaaactgaagctatatttttcacagaagaagaagcagtgacggggacacaaattctgttgcctggtggaaagaaggcaaaggccttcagcaatctatattaccagcgctggatcctttgacagagagtggtccctaaacttaaatttcaagacggtataggcttgatctgtcttgcttattgttgccccctgcgcctagcacaattctgacacacaattggaacttactaaaaatttttttttactgttaaaaaaaaaaaaaaaaaa

SEQ ID NO: 29 - protein, 378 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 30 - protein, 350 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 31 - DNA, 1374 bp, Homo sapiens
ataagaggttgggctttggatagatagacagactcctgggtccggtcaaccgtcaaaatgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgttgagtctacttctgctgacactgtcctgctgacatccaaagagtccaaagtttgggatctcccatcaacatcccacgtgtcaatgtggaaaacgagtgattctgtgtcttctcctcagcctggagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttagattgaggatgggggcatgacactccagtgtcaaaataagtcttagtagatttccttgtttcataaaaaagactcacttattccatggatgtcattgatccaggcttgctttagtttcatgaatgaagggtactttagagaccacaacttctctgtcaaaaa

SEQ ID NO: 32 - DNA, 1290 bp, Homo sapiens
ataagaggttgggctttggatagatagacagactcctgggtccggtcaaccgtcaaaatgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgttgagtctacttctgctgacactgtcctgctgacatccaaagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttagattgaggatgggggcatgacactccagtgtcaaaataagtcttagtagatttccttgtttcataaaaaagactcacttattccatggatgtcattgatccaggcttgctttagtttcatgaatgaagggtactttagagaccacaacttctctgtcaaaaa

SEQ ID NO: 33 - RNA, 19 nt, Artificial Sequence
  siRNA against TIM-1 receptor
aaacucaacuguuccuaca

SEQ ID NO: 34 - RNA, 19 nt, Artificial Sequence
  siRNA against TIM-1 receptor
cggaaggacacacgcuaua

SEQ ID NO: 35 - RNA, 19 nt, Artificial Sequence
  siRNA against TIM-1 receptor
gcagaaacccacccuacga

SEQ ID NO: 36 - RNA, 19 nt, Artificial Sequence
  siRNA against TIM-1 receptor
ggucacgacuacuccaauu

SEQ ID NO: 37 - protein, 359 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

(19) United States
(12) Patent Application Publication
(10) Pub. No.: US 2016/0015808 A1
(43) Pub. Date: Jan. 21, 2016

(54) TAM RECEPTORS AS VIRUS ENTRY COFACTORS

(71) Applicant: INSTITUT NATIONAL DE LA SANTE ET DE LA RECHERCHE MEDICALE (INSERM), Paris (FR)

(72) Inventors: Ali AMARA, Paris (FR); Laurent MEERTENS, Paris (FR)

(73) Assignees: INSTITUT NATIONAL DE LA SANTE ET DE LA RECHERCHE MEDICALE (INSERM), Paris (FR); UNIVERSITE PARIS DIDEROT- PARIS 7, Paris (FR)

(21) Appl. No.: 14/379,914

(22) PCT Filed: Feb. 20, 2013
(86) PCT No.: PCT/EP13/53388

§ 371 Date: Aug. 20, 2014

(30) Foreign Application Priority Data
Feb. 21, 2012 (EP) 12305192.2
Oct. 17, 2012 (EP) 12306283.8

(51) Int. Cl. A61K 39/395; C12N 15/113; A61K 31/7105; A61K 38/17; A61K 45/06; C07K 16/28; A61K 38/45

(52) U.S. Cl. CPC A61K 39/3955; C07K 16/28; C12N 15/1138; A61K 38/45; A61K 38/17; A61K 45/06; A61K 31/7105; C07K 2317/76

(57) ABSTRACT

The present invention concerns the use of an inhibitor of an interaction between phosphatidylserine and a TAM receptor for preventing or treating a virus entry cofactors, in particular phosphatidylserine harboring virus infection such as flavivirus infection.

### FIELD OF THE INVENTION

The present invention concerns the use of an inhibitor of an interaction between phosphatidylserine and a TAM receptor for preventing or treating a viral infection.

### BACKGROUND TO THE INVENTION

Viral infections are a major threat to public health. The emergence and expansion of life-threatening diseases caused by viruses (e.g. hemorrhagic fever and encephalitis), together with unmet conventional prevention approaches (e.g., vaccines) highlights the necessity of exploring new strategies that target these deadly pathogens.

The Flavivirus genus for example encompasses over 70 small-enveloped viruses containing a single positive-stranded RNA genome. Several members of this genus such as Dengue virus (DV), Yellow Fever Virus (YFV), and West Nile virus (WNV), are mosquito-borne human pathogens causing a variety of medically relevant human diseases including hemorrhagic fever and encephalitis (Gould and Solomon, 2008, Lancet, 371:200-509; Gubler et al., 2007, Fields Virology, 5^{th} Edition, 1153-1252). Dengue disease, which is caused by four antigenically related serotypes (DV1 to DV4), has emerged as a global health problem during the last decades and is one of the most medically relevant arboviral diseases. It is estimated that 50-100 million dengue cases occur annually and more than 2.5 billion people being at risk of infection. Infection by any of the four serotypes causes diseases, ranging from mild fever to life-threatening dengue hemorrhagic fever (DHF) and dengue shock syndrome (DSS). Despite the importance and increasing incidence of DV as a human pathogen, there is currently no licensed vaccine available against DV and the lack of anti-viral drugs severely restricts therapeutic options.

Future efforts to combat dengue disease require a better understanding of the DV life cycle. DV entry into target cells is a promising target for preventive as well as therapeutic anti-viral strategies since it is a major determinant of the host-range, cellular tropism and viral pathogenesis. During primary infection, DV enter host cells by clathrin-mediated endocytosis, a process driven by the interaction between the viral glycoprotein (E protein) with cellular receptors. Within the endosome, the acidic environment triggers an irreversible trimerization of the E protein that results in fusion of the viral and cell membranes, allowing the release of the viral capsid and genomic RNA into the cytosol. To date, the molecular bases of DV-host interactions leading to virus entry are poorly understood and little is known about the identity of the DV cellular receptor(s). DV is known to infect a wide range of cell types. DV may thus exploit different receptors, depending on the target cell, or use widely expressed entry molecules. Earlier studies indicated that DV virions make initial contact with the host by binding to heparan-sulfate proteoglycans on the cell membrane. These molecules recognize the positively charged residues on the surface of E protein and are thought to concentrate the virus at the target cell surface before its interactions with entry factors. Numerous cellular proteins such as heat shock protein 70 (HSP70), HSP90, GRP78/Bip, a lipopolysaccharide receptor-CD14 or the 37/67 kDa high affinity laminin have been proposed as putative DV entry receptors. However, their function in viral entry remains poorly characterized and of unclear physiological relevance. To date, the only well-characterized factors that actively participate in the DV entry program are DC-SIGN expressed on dendritic cells, L-SIGN expressed on liver sinusoidal endothelial cells and the mannose receptor (MR) expressed on macrophages. These molecules belong to the C-type lectin receptor family and bind mannose-rich N-linked glycans expressed on the DV E protein. However, DV infects cell types that do not express DC-SIGN, MR or L-SIGN, indicating that other relevant entry receptor(s) exist and remain to be identified.

In addition to the classical cell entry pathway, which is mediated by E protein interaction with cell surface receptors, it is plausible that soluble components present in body fluids and tissues may interact with DV particles and enhance virus internalization. In support of this hypothesis, a recent study indicated that uptake of WNV into mosquito cells is mediated by secreted C-type lectin mosGTLC-1. This soluble factor binds WNV particles with high affinity to putatively bridging them to its cellular receptor mosPTP-1, thus facilitating virus entry. Another example is represented by human adenovirus (HAdV)V5, which interacts with human blood coagulation factor X (FX), resulting in the formation of FX-HAdV5 complexes that are the major parameter responsible for the massive liver uptake of HAdV5 vector particles. Based on the above information, it is likely that the process of DV entry is probably more complex than previously thought. It is reasonable to postulate that attachment and internalization of DV are multistep processes that engage several receptors and possibly soluble components circulating in body fluids that might favor cellular binding and viral tropism of DV.

Currently, DV has become a global problem and is endemic in more than 110 countries. Thus, development of a prophylactic or curative treatment DV infection is needed.

Moreover, deciphering the mechanism of DV internalization might also pave the way to developing treatment of other viral infections.

### DESCRIPTION OF THE INVENTION

The inventors have found that DV infection is mediated by interaction between phosphatidylserine (PtdSer) present at the surface of the DV viral envelope and TAM receptor present at the surface of the host cell, and that such interaction can be blocked, thereby inhibiting entry of DV into host cells and preventing DV infection.

Furthermore, the inventors found that this interaction between phosphatidylserine (PtdSer) and TAM receptors is not only used by other flavivirus such as Yellow Fever Virus (YFN) and West Nile Virus (WNV) but also for example by the Chikungunya Virus showing that this interaction may represent a general mechanism exploited by viruses that incorporate phosphatidylserine (PtdSer) in their membrane.

Thus, the invention relates to an inhibitor of an interaction between phosphatidylserine and a TAM receptor for use for preventing or treating a viral infection, in particular a phosphatidylserine (PtdSer) harboring virus infection such as a flavivirus infection, wherein said inhibitor is preferably (i) a TAM receptor inhibitor, (ii) a Gas6 inhibitor, and/or (iii) a phosphatidylserine binding protein. Preferably, said interaction is an indirect interaction. By “a phosphatidylserine harboring virus infection” is meant in particular a “flavivirus infection”. By “flavivirus infection” it is meant an infection with a Dengue virus (DV), a West Nile virus, a tick-borne encephalitis virus, a Saint-Louis encephalitis virus, a Japanese encephalitis virus or a yellow fever virus. Preferably, said TAM receptor is TYRO3, AXL or MER. Preferably, said TAM receptor inhibitor is an anti-TAM receptor antibody, an antisense nucleic acid, a mimetic or a variant TAM receptor, and more preferably said TAM receptor inhibitor is a siRNA. Preferably, said Gas6 inhibitor is an anti-Gas6 antibody, an antisense nucleic acid, a mimetic or a variant Gas6 protein. Preferably, said phosphatidylserine binding protein is an anti-phosphatidylserine antibody or Annexin 5.

Also provided is a pharmaceutical composition comprising an inhibitor of an interaction between phosphatidylserine and a TAM receptor and additionally at least one other antiviral compound. Preferably, said at least one other antiviral compound is an inhibitor of an interaction of phosphatidylserine and a TIM receptor.

Further provided is the use of an inhibitor of an interaction between phosphatidylserine and a TAM receptor in a method of inhibiting entry of a virus, in particular a PtdSer harboring virus such as a flavivirus into a cell.

Also provided is a method for preventing or treating a viral infection, in particular a PtdSer harboring virus infection such as a flavivirus infection comprising administering to an individual in need thereof a therapeutically effective amount of an inhibitor of an interaction between phosphatidylserine and a TAM receptor.

Also provided is the use of an inhibitor of an interaction between phosphatidylserine and a TAM receptor for the manufacture of a medicament for preventing or treating a viral infection, in particular a PtdSer harboring virus infection, in particular a flavivirus infection.

### Definitions

By “a phosphatidylserine harboring virus infection” is meant an infection with an enveloped virus that expresses or incorporates PtdSer in its membrane. Prior to infection, the PtdSer is exposed on the viral membrane to receptors of the host cell. Examples of enveloped viruses harboring PtdSer include, but are not limited to: Flavivirus (such as Dengue Virus, West Nile Virus, Yellow Fever Virus), Alphavirus (e.g. Chikungunya Virus), Filovirus (e.g. Ebola Virus), Poxivirus (e.g. Cowpox Virus) and Arenavirus (e.g. Lassa Virus).
“A phosphatidylserine harboring virus infection” may include, for example, a “flavivirus infection”. By “flavivirus infection” it is meant an infection with a Dengue virus (DV), a West Nile virus, a tick-borne encephalitis virus, a Saint-Louis encephalitis virus, a Japanese encephalitis virus or a yellow fever virus (Sabin et al., 1952, A.B. Am. J. Trop. Med. Hyg. 1:30-50; Hammon et al., 1960, Trans. Assoc. Am. Physicians 73:140-155; Smithburn, 1940, Am. J. Trop. Med., 20:471-492; Monath and Heinz, 1996, Flaviviruses, Fields Virology, 3^{rd} edition, p.961-1034; Gould and Solomon, 2008, Lancet, 371:500-509). The Dengue virus may be of any serotype, i.e. serotype 1, 2, 3 or 4.

By “interaction between phosphatidylserine and a TAM receptor” is preferably meant the indirect interaction between phosphatidylserine present at the surface of the PtdSer harboring and a TAM receptor present at the surface of the host cell. In fact, the inventors have found that the interaction between phosphatidylserine and TAM receptor is mediated by a bridge molecule, which may be for example the Gas6 protein, and that this indirect interaction permits the PtdSer-harboring virus infection or entry into the host cells.

By “inhibitor” is meant an agent that is able to reduce or to abolish the interaction between phosphatidylserine and a TAM receptor. Said inhibitor may also be able to reduce or abolish the expression of a TAM receptor and/or of a bridge molecule, such as Gas6. According to the invention, said inhibitor may be for example (i) a TAM receptor inhibitor, (ii) a Gas6 or other bridge molecule inhibitor, and/or (iii) a phosphatidylserine binding protein.

Preferably, said inhibitor is able to reduce or to abolish the interaction between phosphatidylserine and a TAM receptor, and/or to reduce or abolish the expression of a TAM receptor and/or of a bridge molecule, by at least 10, 20, 30, 40%, more preferably by at least 50, 60, 70%, and most preferably by at least 75, 80, 85, 90, 95, 96, 97, 98, 99, or 100%.

Reference herein to polypeptides and nucleic acid includes both the amino acid sequences and nucleic acid sequences disclosed herein and variants of said sequences.

Variant proteins may be naturally occurring variants, such as splice variants, alleles and isoforms, or they may be produced by recombinant means. Variations in amino acid sequence may be introduced by substitution, deletion or insertion of one or more codons into the nucleic acid sequence encoding the protein that results in a change in the amino acid sequence of the protein. Optionally the variation is by substitution of 1, 2, 3, 4, 5, 6, 7, 8, 9, 10, 11, 12, 13, 14, 15, 16, 17, 18, 19, 20 or more amino acids with any other amino acid in the protein. Additionally or alternatively, the variation may be by addition or deletion of 1, 2, 3, 4, 5, 6, 7, 8, 9, 10, 11, 12, 13, 14, 15, 16, 17, 18, 19, 20 or more amino acids within the protein.

Variant nucleic acid sequences include sequences capable of specifically hybridizing to the sequence of SEQ ID Nos: 1-4, 6, 9, 10, 12, 16-18, 21, 23-25, 28, 31, 32, 33-36 under moderate or high stringency conditions. Stringent conditions or high stringency conditions may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Moderately stringent conditions may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C.

Fragments of the proteins and variant proteins disclosed herein are also encompassed by the invention. Such fragments may be truncated at the N-terminus or C-terminus, or may lack internal residues, for example, when compared with a full length protein. Certain fragments lack amino acid residues that are not essential for enzymatic activity. Preferably, said fragments are at least about 10, 20, 30, 40, 50, 60, 70, 80, 90, 100, 110, 120, 150, 250, 300, 350, 400, 450, 500 or more amino acids in length.

Fragments of the nucleic acid sequences and variants disclosed herein are also encompassed by the invention. Such fragments may be truncated at 3′ or 5′ end, or may lack internal bases, for example, when compared with a full length nucleic acid sequence. Preferably, said fragments are at least about 10, 20, 30, 40, 50, 60, 70, 80, 90, 100, 110, 120, 150, 250, 300, 350, 400, 450, 500 or more bases in length.

Variant proteins may include proteins that have at least about 80% amino acid sequence identity with a polypeptide sequence disclosed herein. Preferably, a variant protein will have at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% amino acid sequence identity to a full-length polypeptide sequence or a fragment of a polypeptide sequence as disclosed herein. Amino acid sequence identity is defined as the percentage of amino acid residues in the variant sequence that are identical with the amino acid residues in the reference sequence, after aligning the sequences and introducing gaps, if necessary, to achieve the maximum percent sequence identity, and not considering any conservative substitutions as part of the sequence identity. Sequence identity may be determined over the full length of the variant sequence, the full length of the reference sequence, or both.

Variant nucleic acid sequences may include nucleic acid sequences that have at least about 80% amino acid sequence identity with a nucleic acid sequence disclosed herein. Preferably, a variant nucleic acid sequences will have at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% amino acid sequence identity to a full-length nucleic acid sequence or a fragment of a nucleic acid sequence as disclosed herein. Nucleic acid sequence identity is defined as the percentage of nucleic acids in the variant sequence that are identical with the nucleic acids in the reference sequence, after aligning the sequences and introducing gaps, if necessary, to achieve the maximum percent sequence identity, and not considering any conservative substitutions as part of the sequence identity. Sequence identity may be determined over the full length of the variant sequence, the full length of the reference sequence, or both.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, or substituted with another amino acid.

In the context of the present application, the percentage of identity is calculated using a global alignment (i.e. the two sequences are compared over their entire length).

Methods for comparing the identity of two or more sequences are well known in the art. The <<needle>> program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS::needle (global) program with a “Gap Open” parameter equal to 10.0, a “Gap Extend” parameter equal to 0.5, and a Blosum62 matrix.

Proteins consisting of an amino acid sequence “at least 80%, 85%, 90%, 95%, 96%, 97%, 98% or 99% identical” to a reference sequence may comprise mutations such as deletions, insertions and/or substitutions compared to the reference sequence. In case of substitutions, the protein consisting of an amino acid sequence at least 80%, 85%, 90%, 95%, 96%, 97%, 98% or 99% identical to a reference sequence may correspond to a homologous sequence derived from another species than the reference sequence.

Amino acid substitutions may be conservative or non-conservative. Preferably, substitutions are conservative substitutions, in which one amino acid is substituted for another amino acid with similar structural and/or chemical properties. The substitution preferably corresponds to a conservative substitution as indicated in the table below.

| Conservative substitutions | Type of Amino Acid |
|---|---|
| Ala, Val, Leu, Ile, Met, Pro, Phe, | Amino acids with aliphatic |
| Trp | hydrophobic side chains |
| Ser, Tyr, Asn, Gln, Cys | Amino acids with uncharged |
| | but polar side chains |
| Asp, Glu | Amino acids with acidic side chains |
| Lys, Arg, His | Amino acids with basic side chains |
| Gly | Neutral side chain |

The term “antibody” refers to immunoglobulin molecules and immunologically active portions of immunoglobulin molecules, i.e., molecules that contain an antigen binding site which immunospecifically binds an antigen. As such, the term antibody encompasses not only whole antibody molecules, but also antibody fragments as well as variants of antibodies, including derivatives such as humanized antibodies. In natural antibodies, two heavy chains are linked to each other by disulfide bonds and each heavy chain is linked to a light chain by a disulfide bond. There are two types of light chain, lambda (A) and kappa (K). There are five main heavy chain classes (or isotypes) which determine the functional activity of an antibody molecule: IgM, IgD, IgG, IgA and IgE.

Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non hypervariable or framework regions (FR) influence the overall domain structure and hence the combining site. Complementarity determining regions (CDRs) refer to amino acid sequences which, together, define the binding affinity and specificity of the natural Fv region of a native immunoglobulin binding-site. The light and heavy chains of an immunoglobulin each have three CDRs, designated L-CDR1, L-CDR2, L-CDR3 and H-CDR1, H-CDR2, H-CDR3, respectively. Therefore, an antigen-binding site includes six CDRs, comprising the CDR set from each of a heavy and a light chain V region.

Framework Regions (FRs) refer to amino acid sequences interposed between CDRs, i.e. to those portions of immunoglobulin light and heavy chain variable regions that are relatively conserved among different immunoglobulins in a single species, as defined by Kabat, et al (Sequences of Proteins of Immunological Interest (National Institutes of Health, Bethesda, Md., 1991). As used herein, a “human framework region” is a framework region that is substantially identical (about 85%, or more, in particular 90%, 95%, or 100%) to the framework region of a naturally occurring human antibody.

The term “monoclonal antibody” or “mAb” as used herein refers to an antibody molecule of a single amino acid composition, that is directed against a specific antigen and which may be produced by a single clone of B cells or hybridoma. Monoclonal antibodies may also be recombinant, i.e. produced by protein engineering.

The term “chimeric antibody” refers to an engineered antibody which comprises a VH domain and a VL domain of an antibody derived from a non-human animal, in association with a CH domain and a CL domain of another antibody, in particular a human antibody. As the non-human animal, any animal such as mouse, rat, hamster, rabbit or the like can be used. A chimeric antibody may also denote a multispecific antibody having specificity for at least two different antigens.

The term “humanized antibody” refers to antibodies in which the framework or “complementarity determining regions” (CDR) have been modified to comprise the CDR from a donor immunoglobulin of different specificity as compared to that of the parent immunoglobulin. In a preferred embodiment, a mouse CDR is grafted into the framework region of a human antibody to prepare the “humanized antibody”.

“Antibody fragments” comprise a portion of an intact antibody, preferably the antigen binding or variable region of the intact antibody. Examples of antibody fragments include Fv, Fab, F(ab′)2, Fab′, dsFv, scFv, sc(Fv)2, diabodies and multispecific antibodies formed from antibody fragments.

The term “Fab′” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)₂” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab′” refers to an antibody fragment having a molecular weight of about 50,000 and antigen binding activity, which is obtained by cutting a disulfide bond of the hinge region of the F(ab′)₂.

A single chain Fv (“scFv”) polypeptide is a covalently linked VH::VL heterodimer which is usually expressed from a gene fusion including VH and VL encoding genes linked by a peptide-encoding linker. The human scFv fragment of the invention includes CDRs that are held in appropriate conformation, preferably by using gene recombination techniques. “dsFv” is a VH::VL heterodimer stabilised by a disulphide bond. Divalent and multivalent antibody fragments can form either spontaneously by association of monovalent scFvs, or can be generated by coupling monovalent scFvs by a peptide linker, such as divalent sc(Fv)₂.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993, Nature, 365: 566) interactions and alters the activity of the target RNA (for a review, see Stein and Cheng, 1993, Science, 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop or hairpin, and/or an antisense molecule can bind such that the antisense molecule forms a loop or hairpin. Thus, the antisense molecule can be complementary to 2, 3, 4, 5, 6, 7, 8, 9, 10 or more non-contiguous substrate sequences or 2, 3, 4, 5, 6, 7, 8, 9, 10 or more non-contiguous sequence portions of an antisense molecule can be complementary to a target sequence or both (for example, see Crooke, 2000, Methods Enzymol., 313: 3-45). In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA.

Upon introduction, the antisense nucleic acid enters a cellular pathway that is commonly referred to as the RNA interference (RNAi) pathway. The term “RNA interference” or “RNAi” refers to selective intracellular degradation of RNA also referred to as gene silencing. RNAi also includes translational repression by small interfering RNAs (siRNAs). RNAi can be initiated by introduction of Long double-stranded RNA (dsRNAs) or siRNAs or production of siRNAs intracellularly, eg from a plasmid or transgene, to silence the expression of one or more target genes. Alternatively RNAi occurs in cells naturally to remove foreign RNAs, eg viral RNAs. Natural RNAi proceeds via dicer directed fragmentation of precursor dsRNA which direct the degradation mechanism to other cognate RNA sequences.

In some embodiments, the antisense nucleic acid may be Long double-stranded RNAs (dsRNAs), microRNA (miRNA) and/or small interferent RNA (siRNA).

As used herein “Long double-stranded RNA” or “dsRNA” refers to an oligoribonucleotide or polyribonucleotide, modified or unmodified, and fragments or portions thereof, of genomic or synthetic origin or derived from the expression of a vector, which may be partly or fully double stranded and which may be blunt ended or contain a 5′ and or 3′ overhang, and also may be of a hairpin form comprising a single oligoribonucleotide which folds back upon itself to give a double stranded region. In some embodiments, the dsRNA has a size ranging from 150 bp to 3000 bp, preferably ranging from 250 bp to 2000 bp, still more preferably ranging from 300 bp to 1000 bp. In some embodiments, said dsRNA has a size of at least 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, 900, 950, 1000, 1500 bp. In some embodiments, said dsRNA has a size of at most 3000, 2500, 2000, 1500, 1000, 950, 900, 850, 800, 750, 700, 650, 600, 550, 500, 450, 400, 350, 300 bp.

A “small interfering RNA” or “siRNA” is a RNA duplex of nucleotides that is targeted to a gene interest. A RNA duplex refers to the structure formed by the complementary pairing between two regions of a RNA molecule. siRNA is targeted to a gene in that the nucleotide sequence of the duplex portion of the siRNA is complementary to a nucleotide sequence of the targeted gene. In some embodiments, the length of the duplex of siRNAs is ranging from 15 nucleotides to 50 nucleotides, preferably ranging from 20 nucleotides to 35 nucleotides, still more preferably ranging from 21 nucleotides to 29 nucleotides. In some embodiments, the duplex can be of at least 15, 16, 17, 18, 19, 20, 21, 22, 23, 24, 25, 26, 27, 28, 29, 30, 31, 32, 33, 34, 35, 40, 45, 50 nucleotides in length. In some embodiments, the duplex can be of at most 45, 40, 35, 34, 33, 32, 31, 30, 29, 28, 27, 26, 25, 24, 23, 22, 21, 20, 19, 18, 17, 16, 15 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, or 13 nucleotides in length. The hairpin structure can also contain 3 or 5 overhang portions. In some embodiments, the overhang is a 3′ or a 5′ overhang 0, 1, 2, 3, 4, or 5 nucleotides in length.

Injection and transfection antisense nucleic acid into cells and organisms has been the main method of delivery. However, expression vectors may also be used to continually express antisense nucleic acid in transiently and stably transfected mammalian cells. (See for example, e.g., Brummelkamp et al., 2002, Science, 296:550-553; Paddison et al., 2002, Genes & Dev, 16:948-958).

Antisense nucleic acid may be synthesized chemically or expressed via the use of a single stranded DNA expression vector or equivalent thereof using protocols known in the art as described for example in Caruthers et al., 1992, Methods in Enzymology, 211:3-19; International PCT Publication No. WO 99/54459; Brennan et al., 1998, Biotechnol Bioeng, 61:33-45, and U.S. Pat. No. 6,001,311. In a non-limiting example, small scale syntheses are conducted on a 394 Applied Biosystems, Inc. synthesizer. Alternatively, the antisense nucleic acid molecules of the present invention can be synthesized separately and joined together post-synthetically, for example by ligation (International PCT publication No. WO 93/23569, Bellon et al., 1997, Bioconjugate Chem, 8:204).

The antisense nucleic acid of the invention may be able of decreasing the expression of the targeted gene, for example TAM receptor or Gas6 protein, by at least 10, 20, 30, 40%, more preferably by at least 50, 60, 70%, and most preferably by at least 75, 80, 85, 90, 95, 96, 97, 98, 99, 100%.

By “variant TAM receptor” or “variant Gas6 protein” or “variant TIM receptor” is meant respectively a receptor or a protein that differs from the TAM receptor or the Gas6 protein or the TIM receptor by one or several amino acid(s). For example, said variant TAM receptor may differ from the TAM receptor in that it is no longer able to bind to the Gas6 protein, such as for example an AXL receptor of sequence SEQ ID NO: 7 or 8 carrying the mutation E63R, E66R or T847R, or in that it is no longer able to have its kinase activity, such as for example an AXL receptor of sequence SEQ ID NO: 7 carrying the mutation K558M, or an AXL receptor of sequence SEQ ID NO: 8 carrying the mutation K567M. For example, said variant Gas6 protein may differ from the Gas6 protein in that it is no longer able to bind to phosphatidylserine and/or to a TAM receptor. For example, said variant Gas6 protein may be the Gas6ΔgIa (also named rmGas6ΔgIa) of sequence SEQ ID NO: 19. For example, said variant TIM receptor may differ from the TIM receptor in that it is no longer able to bind to phosphatidylserine or in that it is no longer able to have its kinase activity.

The terms “subject”, “individual” or “host” are used interchangeably and may be, for example, a human or a non-human mammal. For example, the subject is a bat; a ferret; a rabbit; a feline (cat); a canine (dog); a primate (monkey), an equine (horse); a human, including man, woman and child.

Inhibitor of Interaction Between Phosphatidylserine and a TAM Receptor

Phosphatidylserine is a phospholipid which phosphate group is associated to the serine amino acid and which is referenced under the CAS number 8002-43-5.

By “TAM receptor” is meant a tyrosine kinase receptor of the Tyro3/Axl/Mer family. In preferred embodiments, said TAM receptor is a TYRO-3, AXL or MER receptor.

Preferably, the TYRO-3 receptor comprises or consists of:
a) the sequence SEQ ID NO: 5 (NCBI Reference Sequence NP_{—}006284.2, update Nov. 14, 2011),
b) the sequence encoded by the nucleic acid of sequence SEQ ID NO: 6 (NCBI Reference Sequence NM_{—}006293.3, update Jan. 14, 2012),
c) a sequence at least 80, 85, 90, 95, 96, 97, 98, 99% identical to the sequence of a) or b).

Preferably, the AXL receptor comprises or consists of:
a) the sequence SEQ ID NO: 7 (NCBI Reference Sequence NP_{—}001690.2, update Nov. 26, 2011),
b) the sequence SEQ ID NO: 8 (NCBI Reference Sequence NP_{—}068713.2, update Nov. 26, 2011),
c) the sequence encoded by the nucleic acid of sequence SEQ ID NO: 9 (NCBI Reference Sequence NM_{—}021913.3, update Jan. 15, 2012),
d) the sequence encoded by the nucleic acid of sequence SEQ ID NO: 10 (NCBI Reference Sequence NM_{—}001699.4, update Jan. 15, 2012),
e) a sequence at least 80, 85, 90, 95, 96, 97, 98, 99% identical to the sequence of a) to d).

Preferably, the MER receptor comprises or consists of:
a) the sequence SEQ ID NO: 11 (NCBI Reference Sequence NP_{—}006334.2, update Dec. 24, 2011),
b) the sequence encoded by the nucleic acid of sequence SEQ ID NO: 12 (NCBI Reference Sequence NM_{—}006343.2, update Dec. 24, 2011),
c) a sequence at least 80, 85, 90, 95, 96, 97, 98, 99% identical to the sequence of a) or b).

The Gas6 protein is a bridge molecule that mediates the interaction between phosphatidylserine and a TAM receptor.

Preferably, the Gas6 protein comprises or consists of:
a) the sequence SEQ ID NO: 13 (NCBI Reference Sequence NP_{—}000811.1, update Dec. 24, 2011),
b) the sequence SEQ ID NO: 14 (NCBI Reference Sequence NP_{—}001137417.1 update Dec. 24, 2011),
c) the sequence SEQ ID NO: 15 (NCBI Reference Sequence NP_{—}001137418.1, update Dec. 24, 2011),
d) the sequence encoded by the nucleic acid of sequence SEQ ID NO: 16 (NCBI Reference Sequence NM_{—}000820.2, update Jan. 15, 2012),
e) the sequence encoded by the nucleic acid of sequence SEQ ID NO: 17 (NCBI

Reference Sequence NM_{—}001143945.1, update Jan. 15, 2012),
f) the sequence encoded by the nucleic acid of sequence SEQ ID NO: 18 (NCBI Reference Sequence NM_{—}001143946.1, update Jan. 15, 2012),
g) a sequence at least 80, 85, 90, 95, 96, 97, 98, 99% identical to the sequence of a) to f).

In some embodiments, the TAM receptor inhibitor is an anti-TAM receptor antibody, an antisense nucleic acid, a mimetic or a variant TAM receptor.

Preferably, said TAM receptor inhibitor is an antisense nucleic acid, and more preferably said TAM receptor inhibitor is a siRNA. Said antisense nucleic acid may comprise or consist of a sequence that is able to inhibit or reduce the expression of a TAM receptor of sequence SEQ ID NO: 5, 7, 8, or 11, or a TAM receptor of sequence encoded by the nucleic acid SEQ ID NO: 6, 9, 10, or 12. Said antisense nucleic acid may comprise or consist of a sequence complementary to a nucleic acid encoding a TAM receptor or fragment thereof, for example a nucleic acid of sequence SEQ NO: 6, 9, 10, or 12. In one embodiment, said siRNA comprises or consists of at least one siRNA of sequence SEQ ID NO: 1, 2, 3, or 4. In one embodiment, said siRNA comprises or consists of at least 2, 3, or 4 siRNA selected from the group consisting of SEQ ID NOs: 1, 2, 3, and 4. In one embodiment, said siRNA comprises or consists of at most 4, 3, or 2 siRNA selected from the group consisting of SEQ ID NOs: 1, 2, 3, and 4. In one embodiment, said siRNA comprises or consists of the four siRNA of sequence SEQ ID NO: 1, 2, 3, and 4.

Preferably, said mimetic comprises or consists of the extracellular domain of the TAM receptor. For example, said mimetic may comprise or consist of the amino acids 26 to 451 of SEQ ID NO: 7 or SEQ ID NO: 8.

Still more preferably, said mimetic comprises or consists of the soluble form of the extracellular domain of the TAM receptor. For example, said mimetic may comprise or consist of the sequence of amino acids 41 to 428 of SEQ ID NO: 5, or of the sequence of amino acids 33 to 440 of SEQ ID NO: 7 or SEQ ID NO: 8.

Preferably, said anti-TAM receptor antibody is an antibody directed against the binding site of the TAM receptor to the Gas6 protein. Preferably, said anti-TAM receptor antibody is directed to the amino acids 63 to 84 of the sequence SEQ ID NO: 7 or SEQ ID NO: 8.

In some embodiments, the Gas6 inhibitor is an anti-Gas6 antibody, an antisense nucleic acid, a mimetic or a variant Gas6 protein.

Preferably, said Gas6 inhibitor is an antisense nucleic acid, and more preferably said Gas6 inhibitor is a siRNA. Said antisense nucleic acid may comprise or consist of a sequence that is able to inhibit or reduce the expression of a Gas6 protein of sequence SEQ ID NO: 13, 14, or 15, or a Gas6 protein of sequence encoded by the nucleic acid SEQ ID NO: 16, 17, or 18. Said antisense nucleic acid may comprise or consist of a sequence complementary to a nucleic acid encoding Gas6 or fragment thereof, for example a nucleic acid of sequence SEQ NO: 16, 17, or 18.

Preferably, said Gas6 inhibitor is the variant Gas6 protein Gas6ΔGIa of sequence SEQ ID NO: 19.

Preferably, said Gas-6 mimetic comprises or consists of the phosphatidylserine recognition site which may comprise or consist of the amino acid sequence of residues 53 to 94 of SEQ ID NO: 13 or said mimetic comprises or consists of the receptor binding site which may comprise or consist of the amino acid sequence of residues 298 to 670 of SEQ ID NO: 13.

Preferably, said anti-Gas6 antibody is an antibody directed against the binding site of the Gas6 protein to the TAM receptor. Preferably, said anti-Gas6 antibody is directed to the amino acids 304 to 312 of the sequence SEQ ID NO: 13, to the amino acids 31 to 39 of the sequence SEQ ID NO: 14, or to the amino acids 5 to 13 of the sequence SEQ ID NO: 15.

The phosphatidylserine binding protein may be a protein that is able to bind to the phosphatidylserine but that is not able to bind to the Gas6 protein. Preferably, said phosphatidylserine binding protein is an anti-phosphatidylserine antibody or the Annexin V.

Preferably, said anti-phosphatidylserine antibody is an antibody directed against the binding site of the phosphatidylserine to the Gash protein. For example, said antibody may be the anti-phosphatidylserine antibody clone 1 H6 (Upstate®).

Preferably, said Annexin V protein comprises or consists of:
a) the sequence SEQ ID NO: 20 (NCBI Reference Sequence NP_{—}001145.1, update Feb. 1, 2012),
b) the sequence encoded by the nucleic acid of sequence SEQ ID NO: 21 (NCBI Reference Sequence NM_{—}001154.3, update Dec. 18, 2011),
c) a sequence at least 80, 85, 90, 95, 96, 97, 98, 99% identical to the sequence of a) or b).

### Antiviral Compounds

In a preferred embodiment, the inhibitor according to the invention is for administration in combination with at least one other antiviral compound, either sequentially or simultaneously.

Sequential administration indicates that the components are administered at different times or time points, which may nonetheless be overlapping. Simultaneous administration indicates that the components are administered at the same time.

The antiviral compound may include, but is not limited to, neuraminidase inhibitors, viral fusion inhibitors, protease inhibitors, DNA polymerase inhibitors, signal transduction inhibitors, reverse transcriptase inhibitors, interferons, nucleoside analogs, integrase inhibitors, thymidine kinase inhibitors, viral sugar or glycoprotein synthesis inhibitors, viral structural protein synthesis inhibitors, viral attachment and adsorption inhibitors, viral entry inhibitors and their functional analogs.

Neuraminidase inhibitors may include oseltamivir, zanamivir and peramivir. Viral fusion inhibitors may include cyclosporine, maraviroc, enfuviritide and docosanol.

Protease inhibitors may include saquinavir, indinarvir, amprenavir, nelfinavir, ritonavir, tipranavir, atazanavir, darunavir, zanamivir and oseltamivir.

DNA polymerase inhibitors may include idoxuridine, vidarabine, phosphonoacetic acid, trifluridine, acyclovir, forscarnet, ganciclovir, penciclovir, cidoclovir, famciclovir, valaciclovir and valganciclovir.

Signal transduction inhibitors include resveratrol and ribavirin. Nucleoside reverse transcriptase inhibitors (NRTIs) may include zidovudine (ZDV, AZT), lamivudine (3TC), stavudine (d4T), zalcitabine (ddC), didanosine (2′,3′-dideoxyinosine, ddI), abacavir (ABC), emirivine (FTC), tenofovir (TDF), delaviradine (DLV), fuzeon (T-20), indinavir (IDV), lopinavir (LPV), atazanavir, combivir (ZDV/3TC), kaletra (RTV/LPV), adefovir dipivoxil and trizivir (ZDV/3TC/ABC). Non-nucleoside reverse transcriptase inhibitors (NNRTIs) may include nevirapine, delavirdine, UC-781 (thiocarboxanilide), pyridinones, TIBO, calanolide A, capravirine and efavirenz.

Viral entry inhibitors may include Fuzeon (T-20), NB-2, NB-64, T-649, T-1249, SCH-C, SCH-D, PRO 140, TAK 779, TAK-220, RANTES analogs, AK602, UK-427, 857, monoclonal antibodies against relevant receptors, cyanovirin-N, clyclodextrins, carregeenans, sulfated or sulfonated polymers, mandelic acid condensation polymers, AMD-3100, and functional analogs thereof.

Preferably, said at least one other antiviral compound is an inhibitor of an interaction between phosphatidylserine and a TIM receptor.

By “TIM receptor”, it is meant a TIM-1, TIM-3 or TIM-4 receptor.

In some embodiments, the TIM-1 receptor comprises or consists of:
a) the sequence SEQ ID NO: 22 (GenBank Number AAH13325.1, update Oct., 4, 2003),
b) the sequence encoded by the nucleic acid SEQ ID NO: 23 (NCBI Reference Sequence NM_{—}012206.2, update Nov. 26, 2011),
c) the sequence encoded by the nucleic acid SEQ ID NO: 24 (NCBI Reference Sequence NM_{—}001099414.1, update Nov. 26, 2011),
d) the sequence encoded by the nucleic acid SEQ ID NO: 25 (NCBI Reference Sequence NM_{—}001173393.1, update Dec. 4, 2011),
e) a sequence at least 80, 85, 90, 95, 96, 97, 98, 99% identical to the sequence of a) to d).

In some embodiments, the TIM-3 receptor comprises or consists of:
a) the sequence SEQ ID NO: 26 (GenBank Number AAH20843.1, update Sep. 16, 2003),
b) the sequence SEQ ID NO: 27 (GenBank Number AAH63431.1, update Jul. 15, 2006),
c) the sequence encoded by the nucleic acid SEQ ID NO: 28 (NCBI Reference Sequence NM_{—}032782.4, update Dec. 25, 2011),
d) a sequence at least 80, 85, 90, 95, 96, 97, 98, 99% identical to the sequence of a) to c).

In some embodiments, the TIM-4 receptor comprises or consists of:
a) the sequence SEQ ID NO: 29 (NCBI Reference Sequence NP_{—}612388.2, update Dec. 24, 2011),
b) the sequence SEQ ID NO: 30 (NCBI Reference Sequence NP_{—}001140198.1, update Dec. 25, 2011),
c) the sequence encoded by the nucleic acid SEQ ID NO: 31 (NCBI Reference Sequence NM_{—}138379.2, update Dec. 24, 2011),
d) the sequence encoded by the nucleic acid SEQ ID NO: 32 (NCBI Reference Sequence NM_{—}001146726.1, update Dec. 25, 2011),
e) a sequence at least 80, 85, 90, 95, 96, 97, 98, 99% identical to the sequence of a) to d).

In some embodiments, said inhibitor of interaction of phosphatidylserine and a TIM receptor is a TIM receptor inhibitor. Preferably said TIM receptor inhibitor is an anti-TIM receptor antibody, an antisense nucleic acid, a mimetic or a variant TIM receptor. Preferably, said TIM receptor inhibitor is an antisense nucleic acid, and more preferably said TIM receptor inhibitor is a siRNA. Said antisense nucleic acid may comprise or consist of a sequence that is able to inhibit or reduce the expression of a TIM receptor of sequence SEQ ID NO: 22, 26, 27, 29, or 30, or a TIM receptor of sequence encoded by the nucleic acid SEQ ID NO: 23, 24, 25, 28, 31, or 32. Said antisense nucleic acid may comprise or consist of a sequence complementary to a nucleic acid encoding a TIM receptor, for example a nucleic acid of sequence SEQ NO: 23, 24, 25, 28, 31, or 32. In one embodiment, said TIM receptor inhibitor comprises or consists of at least one siRNA of sequence SEQ ID NO: 33, 34, 35 or 36. In one embodiment, said siRNA comprises or consists of at least 2, 3, or 4 siRNA selected from the group consisting of SEQ ID NOs: 33, 34, 35, and 36. In one embodiment, said siRNA comprises or consists of at most 4, 3, 2, or 1 siRNA selected from the group consisting of SEQ ID NOs: 33, 34, 35, and 36. In one embodiment, said siRNA comprises or consists of the four siRNA of sequence SEQ ID NO: 33, 34, 35, and 36.

Preferably, said anti-TIM receptor antibody is the anti-TIM1 receptor antibody ARD5 described in Kondratowicz et al., 2011, PNAS, 108:8426-8431, or the anti-TIM1 antibody A6G2 described in Sonar et al., 2010, The Journal of Clinical investigation, 120: 2767-2781.

Preferably, said mimetic comprises or consists of the extracellular domain of the TIM receptor. For example, said mimetic may comprise or consist of the amino acid sequence of residues 21 to 295 for TIM-1 of SEQ ID NO: 22, said mimetic may comprise or consist of the amino acid sequence of residues 21 to 290 for TIM-1 of SEQ ID NO: 37 or said mimetic may comprise or consist of the amino acid sequence of residues 25 to 314 for TIM-4 of SEQ ID NO: 29.

Preferably, said anti-TIM receptor antibody is an antibody directed against the binding site of the TIM receptor to the phosphatidylserine. Preferably, said antibody directed against the binding site of the TIM receptor to phosphatidylserine is directed to the Metal Ion-dependent Ligand Binding Site (MILIB) of the TIM receptor. Still more preferably, said anti-TIM receptor is directed to the amino acids 111 to 115 of sequence SEQ ID NO: 22, or to the amino acids 119 to 122 of sequence SEQ ID NO: 29 or SEQ ID NO: 30.

### Method for Inhibiting Entry of a Phosphatidylserine Harboring Virus into a Cell

The inhibitor according to the invention may be used in a method of inhibiting entry of a PtdSer harboring virus_into a cell.

Said method may be an in vitro or ex vivo method, or a method of prevention or treatment of a PtdSer harboring virus infection as described herein.

The invention thus provides the use of an inhibitor as defined herein in an in vitro or in vivo method for inhibiting entry of a PtdSer harboring virus_into a cell. Also provided is an inhibitor as defined herein for use in an in vitro or in vivo method for inhibiting entry of a PtdSer harboring virus_into a cell.

In some embodiments, said inhibitor is use in combination with at least one other antiviral compound as defined hereabove.

Said method may comprise, for example, exposing said cell and/or said PtdSer harboring virus to said inhibitor. Where the method is an in vivo method, the method may comprise administering said inhibitor to a subject, preferably a patient in need thereof.

In some embodiments, said cell may be dendritic cells, endothelial cells, astrocytes, hepatocytes, neurons, Kupffer cells, and/or macrophages.

### Pharmaceutical Compositions

The inhibitor according to the invention may be formulated in a pharmaceutically acceptable composition, either alone or in combination with the at least one other antiviral compound.

The invention thus provides a pharmaceutical composition comprising an inhibitor according to the invention and additionally at least one other antiviral compound.

Said at least one other antiviral compound may be a compound as defined above.

In one embodiment, said inhibitor comprises or consists of at least 1, 2, 3, or 4, or at most 4, 3, 2, or 1 siRNA selected from the group consisting of siRNA of sequence SEQ ID NOs: 1, 2, 3, and 4, and/or annexin V as defined hereabove, and the at least one other antiviral compound comprises or consists of at least 1, 2, 3, or 4, or at most 4, 3, 2, or 1 siRNA selected from the group consisting of siRNA of sequence SEQ ID NOs: 33, 34, 35, and 36 and/or the variant Gas6 protein Gas6ΔgIa of sequence SEQ ID NO: 19 as defined hereabove. In one embodiment, said inhibitor comprises or consists of 4 siRNA of sequence SEQ ID NOs: 1, 2, 3, and 4, and/or annexin V as defined hereabove, and the at least one other antiviral compound comprises or consists of 4 siRNA of sequence SEQ ID NOs: 33, 34, 35, and 36 and/or the variant Gas6 protein Gas6ΔgIa of sequence SEQ ID NO: 19 as defined hereabove.

The pharmaceutical compositions according to the invention may be administered orally in the form of a suitable pharmaceutical unit dosage form. The pharmaceutical compositions of the invention may be prepared in many forms that include tablets, hard or soft gelatin capsules, aqueous solutions, suspensions, and liposomes and other slow-release formulations, such as shaped polymeric gels.

The mode of administration and dosage forms are closely related to the properties of the therapeutic agents or compositions which are desirable and efficacious for the given treatment application. Suitable dosage forms include, but are not limited to, oral, intravenous, rectal, sublingual, mucosal, nasal, ophthalmic, subcutaneous, intramuscular, transdermal, spinal, intrathecal, intra-articular, intra-arterial, sub-arachnoid, bronchial, and lymphatic administration, and other dosage forms for systemic delivery of active ingredients.

Pharmaceutical compositions of the invention may be administered by any method known in the art, including, without limitation, transdermal (passive via patch, gel, cream, ointment or iontophoretic); intravenous (bolus, infusion); subcutaneous (infusion, depot); transmucosal (buccal and sublingual, e.g., orodispersible tablets, wafers, film, and effervescent formulations; conjunctival (eyedrops); rectal (suppository, enema)); or intradermal (bolus, infusion, depot).

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

Pharmaceutical compositions of the invention may also be formulated for parenteral administration (e.g., by injection, for example, bolus injection or continuous infusion) and may be presented in unit dosage form in ampoules, pre-filled syringes, small volume infusion containers or multi-dose containers with an added preservative. The pharmaceutical compositions may take such forms as suspensions, solutions, or emulsions in oily or aqueous vehicles, and may contain formulating agents such as suspending, stabilizing and/or dispersing agents. Alternatively, the pharmaceutical compositions of the invention may be in powder form, obtained by aseptic isolation of sterile solid or by lyophilization from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the pharmaceutical composition with the softened or melted carrier(s) followed by chilling and shaping in molds.

For administration by inhalation, the pharmaceutical compositions according to the invention are conveniently delivered from an insufflator, nebulizer or a pressurized pack or other convenient means of delivering an aerosol spray. Pressurized packs may comprise suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the pharmaceutical compositions of the invention may take the form of a dry powder composition, for example, a powder mix of the pharmaceutical composition and a suitable powder base such as lactose or starch. The powder composition may be presented in unit dosage form in, for example, capsules or cartridges or, e.g., gelatin or blister packs from which the powder may be administered with the aid of an inhalator or insufflator.

For intra-nasal administration, the pharmaceutical compositions of the invention may be administered via a liquid spray, such as via a plastic bottle atomizer. Typical of these are the Mistometerg (isoproterenol inhaler-Wintrop) and the Medihaler® (isoproterenol inhaler-Riker).

For antisense nucleic acid administration, the pharmaceutical compositions of the invention may be prepared in forms that include encapsulation in liposomes, microparticles, microcapsules, lipid-based carrier systems. Non limiting examples of alternative lipid based carrier systems suitable for use in the present invention include polycationic polymer nucleic acid complexes (see, e.g. US Patent Publication No 20050222064), cyclodextrin polymer nucleic acid complexes (see, e.g. US Patent Publication No 20040087024), biodegradable poly 3 amino ester polymer nucleic acid complexes (see, e.g. US Patent Publication No 20040071654), pH sensitive liposomes (see, e.g. US Patent Publication No 20020192274), anionic liposomes (see, e.g. US Patent Publication No 20030026831), cationic liposomes (see, e.g. US Patent Publication No 20030229040), reversibly masked lipoplexes (see, e.g. US Patent Publication No 20030180950), cell type specific liposomes (see, e.g. US Patent Publication No 20030198664), microparticles containing polymeric matrices (see, e.g. US Patent Publication No 20040142475), pH sensitive lipoplexes (see, e.g. US Patent Publication No 20020192275), liposomes containing lipids derivatized with releasable hydrophilic polymers (see, e.g. US Patent Publication No 20030031704), lipid en trapped nucleic acid (see, e.g. PCT Patent Publication No WO 03/057190), lipid encapsulated nucleic acid (see, e.g. US Patent Publication No 20030129221), polycationic sterol derivative nucleic acid complexes (see, e.g. U.S. Pat. No. 6,756,054), other liposomal compositions (see, e.g. US Patent Publication No 20030035829), other microparticle compositions (see, e.g. US Patent Publication No 20030157030), poly-plexes (see, e.g. PCT Patent Publication No WO 03/066069), emulsion compositions (see, e.g. US Pat No 6,747,014), condensed nucleic acid complexes (see, e.g. US Patent Publication No 20050123600), other polycationic nucleic acid complexes (see, e.g. US Patent Publication No 20030125281), polyvinylether nucleic acid complexes (see, e.g. US Patent Publication No 20040156909), polycyclic amidinium nucleic acid complexes (see, e.g. US Patent Publication No 20030220289), nanocapsule and microcapsule compositions (see, e.g. PCT Patent Publication No WO 02/096551), stabilized mixtures of liposomes and emulsions (see, e.g. EP1304160), porphyrin nucleic acid complexes (see, e.g. U.S. Pat. No. 6,620,805), lipid nucleic acid complexes (see, e.g. US Patent Publication No 20030203865), nucleic acid micro emulsions (see, e.g. US Patent Publication No 20050037086), and cationic lipid based compositions (see, e.g. US Patent Publication No 20050234232). One skilled in the art will appreciate that modified siRNA of the present invention can also be delivered as a naked siRNA molecule.

Pharmaceutical compositions of the invention may also contain other adjuvants such as flavorings, colorings, anti-microbial agents, or preservatives.

It will be further appreciated that the amount of the pharmaceutical compositions required for use in treatment will vary not only with the therapeutic agent selected but also with the route of administration, the nature of the condition being treated and the age and condition of the patient and will be ultimately at the discretion of the attendant physician or clinician.

### Administration and Methods of Treatment

The invention also relates to a method for preventing or treating a PtdSer harboring virus infection in an individual in need thereof comprising administering a therapeutically effective amount of an inhibitor according to the invention.

By “treatment” is meant a therapeutic use (i.e. on a patient having a given disease) and by “preventing” is meant a prophylactic use (i.e. on an individual susceptible of developing a given disease). The term “treatment” not only includes treatment leading to complete cure of the disease, but also treatments slowing down the progression of the disease and/or prolonging the survival of the patient.

An “effective amount” refers to an amount effective, at dosages and for periods of time necessary, to achieve the desired therapeutic or prophylactic result.

A therapeutically effective amount of an inhibitor of the invention may vary according to factors such as the disease state, age, sex, and weight of the individual, and the ability of the protein, to elicit a desired therapeutic result. A therapeutically effective amount encompasses an amount in which any toxic or detrimental effects of the inhibitor are outweighed by the therapeutically beneficial effects. A therapeutically effective amount also encompasses an amount sufficient to confer benefit, e.g., clinical benefit.

In the context of the present invention, “preventing a phosphatidylserine harboring virus infection” may mean prevention of a PtdSer harboring virus infection or entry into the host cell.

In the context of the present invention, “treating a phosphatidylserine harboring virus infection”, may mean reversing, alleviating, or inhibiting phosphatidylserine harboring virus infection or entry into the host cell.

In the context of the invention, phosphatidylserine harboring virus infection may be reduced by at least 30, 35, 40, 45, 50, 55, 60, 65, 70, 80, 85, 90, 91, 92, 93, 94, 95, 96, 97, 98, 99, 100%.

In some embodiments, the methods of the invention comprise the administration of an inhibitor as defined above, in combination with at least one other antiviral compound as defined above, either sequentially or simultaneously. For example, said at least one other antiviral compound is an inhibitor of an interaction between phosphatidylserine and a TIM receptor as defined hereabove.

In another embodiment, said method comprises the administration of a pharmaceutical composition according to the invention.

The administration regimen may be a systemic regimen. The mode of administration and dosage forms are closely related to the properties of the therapeutic agents or compositions which are desirable and efficacious for the given treatment application. Suitable dosage forms and routes of administration include, but are not limited to, oral, intravenous, rectal, sublingual, mucosal, nasal, ophthalmic, subcutaneous, intramuscular, transdermal, spinal, intrathecal, intra-articular, intra-arterial, sub-arachnoid, bronchial, and lymphatic administration, and/or other dosage forms and routes of administration for systemic delivery of active ingredients. In a preferred embodiment, the dosage forms are for parenteral administration.

The administration regimen may be for instance for a period of at least 5, 6, 7, 8, 9, 10, 15, 20, 25, 30, 35, 40, 45, 50, 55, 60, 65, 70, 75, 80, 85, 90, 95, 100 days.

The dose range may be between 0.1 mg/kg/day and 100 mg/kg/day. More preferably, the dose range is between 0.5 mg/kg/day and 100 mg/kg/day. Most preferably, the dose range is between 1 mg/kg/day and 80 mg/kg/day. Most preferably, the dose range is between 5 mg/kg/day and 50 mg/kg/day, or between 10 mg/kg/day and 40 mg/kg/day.

In some embodiments, the dose may be of at least 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 35, 40, 45, 50 mg/kg/day. In some embodiments, the dose may be of at most 50, 45, 40, 35, 30, 25, 20, 25, 15, 10, 5, 1, 0.5, 0.1 mg/kg/day.

The dose range may also be between 10 to 10000 UI/kg/day. More preferably, the dose range is between 50 to 5000 UI/kg/day, or between 100 to 1000 UI/kg/day.

In some embodiments, the dose may be of at least 10, 25, 50, 75, 100, 150, 200, 250, 300, 350, 400, 450, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000 UI/kg/day. In some embodiments, the dose may be of at most 10000, 9500, 9000, 8500, 8000, 7500, 7000, 6500, 6000, 5500, 5000, 4500, 4000, 3500, 3000, 2500, 2000, 1500, 1000, 900, 800, 600, 500, 450, 400, 350, 300, 250, 200, 150, 100 UI/kg/day.

The invention will now be described in more detail with reference to the following figures and examples. All literature and patent documents cited herein are hereby incorporated by reference.

### SEQUENCE LISTING

SEQ ID NO: 1 shows the sequence of the siRNA 5′-ACAGCGAGAUUUAUGACUA-3′ against AXL.
SEQ ID NO: 2 shows the sequence of the siRNA 5′-GGUACCGGCUGGCGUAUCA-3′ against AXL.
SEQ ID NO: 3 shows the sequence of the siRNA 5′-GACGAAAUCCUCUAUGUCA-3′ against AXL.
SEQ ID NO: 4 shows the sequence of the siRNA 5′-GAAGGAGACCCGUUAUGGA-3′ against AXL.
SEQ ID NO: 5 shows the amino acid sequence of TYRO-3 receptor referenced under the NCBI Reference Sequence NP_{—}006284.2.
SEQ ID NO: 6 shows the nucleic acid sequence of TYRO-3 receptor referenced under the NCBI Reference Sequence NM_{—}006293.3.
SEQ ID NO: 7 shows the amino acid sequence of AXL receptor referenced under the NCBI Reference Sequence NP_{—}001690.2.
SEQ ID NO: 8 shows the amino acid sequence of AXL receptor referenced under the NCBI Reference Sequence NP_{—}068713.2.
SEQ ID NO: 9 shows the nucleic acid sequence of AXL receptor referenced under the NCBI Reference Sequence NM_{—}021913.3.
SEQ ID NO: 10 shows the nucleic acid sequence of AXL receptor referenced under the NCBI Reference Sequence NM_{—}001699.4.
SEQ ID NO: 11 shows the amino acid sequence of MER receptor referenced under the NCBI Reference Sequence NP_{—}006334.2.
SEQ ID NO: 12 shows the nucleic acid sequence of MER receptor referenced under the NCBI Reference Sequence NM_{—}006343.2.
SEQ ID NO: 13 shows the amino acid sequence of Gas6 protein referenced under the NCBI Reference Sequence NP_{—}000811.1.
SEQ ID NO: 14 shows the amino acid sequence of Gas6 protein referenced under the NCBI Reference Sequence NP_{—}001137417.1.
SEQ ID NO: 15 shows the amino acid sequence of Gas6 protein referenced under the NCBI Reference Sequence NP_{—}001137418.1.
SEQ ID NO: 16 shows the nucleic acid sequence of Gas6 protein referenced under the NCBI Reference Sequence NM_{—}000820.2.
SEQ ID NO: 17 shows the nucleic acid sequence of Gas6 protein referenced under the NCBI Reference Sequence NM_{—}001143945.1.
SEQ ID NO: 18 shows the nucleic acid sequence of Gas6 protein referenced under the NCBI Reference Sequence NM_{—}001143946.1.
SEQ ID NO: 19 shows the sequence of the variant Gas6ΔGIa protein.
SEQ ID NO: 20 shows the amino acid sequence of Annexin 5 referenced under the NCBI Reference Sequence NP_{—}001145.1.
SEQ ID NO: 21 shows the nucleic acid sequence of Annexin 5 referenced under the NCBI Reference Sequence NM_{—}001154.3.
SEQ ID NO: 22 shows the amino acid sequence of TIM-1 receptor referenced under the GenBank Number AAH13325.1.
SEQ ID NO: 23 shows the nucleic acid sequence of TIM-1 receptor referenced under the NCBI Reference Sequence NM_{—}012206.2.
SEQ ID NO: 24 shows the nucleic acid sequence of TIM-1 receptor referenced under the NCBI Reference Sequence NM_{—}001099414.1.
SEQ ID NO: 25 shows the nucleic acid sequence of TIM-1 receptor referenced under the NCBI Reference Sequence NM_{—}001173393.1.
SEQ ID NO: 26 shows the amino acid sequence of TIM-3 receptor referenced under the GenBank Number AAH20843.1.
SEQ ID NO: 27 shows the amino acid sequence of TIM-3 receptor referenced under the GenBank Number AAH63431.1.
SEQ ID NO: 28 shows the nucleic acid sequence of TIM-3 receptor referenced under the NCBI Reference Sequence NM_{—}032782.4.
SEQ ID NO: 29 shows the amino acid sequence of TIM-4 receptor referenced under the NCBI Reference Sequence NP_{—}612388.2.
SEQ ID NO: 30 shows the amino acid sequence of TIM-4 receptor referenced under the NCBI Reference Sequence NP_{—}001140198.1.
SEQ ID NO: 31 shows the nucleic acid sequence of TIM-4 receptor referenced under the NCBI Reference Sequence NM_{—}138379.2.
SEQ ID NO: 32 shows the nucleic acid sequence of TIM-4 receptor referenced under the NCBI Reference Sequence NM_{—}001146726.1.
SEQ ID NO: 33 shows the sequence of the siRNA 5′-AAACUCAACUGUUCCUACA-3′ against TIM-1.
SEQ ID NO: 34 shows the sequence of the siRNA 5′-CGGAAGGACACACGCUAUA-3′ against TIM-1.
SEQ ID NO: 35 shows the sequence of the siRNA 5′-GCAGAAACCCACCCUACGA-3′ against TIM-1.
SEQ ID NO: 36 shows the sequence of the siRNA 5′-GGUCACGACUACUCCAAUU-3′ against TIM-1.
SEQ ID NO: 37 shows the amino acid sequence of TIM-1 receptor referenced under the UniProt Number Q96D42.

### FIGURES

FIG. 1. TYRO3 and AXL ectopic expression enhance DV Infection. Parental, TYRO3 and AXL-expressing 293T were incubated with DV2 JAM (MOI of 10) for 3 hours. Supernatants were collected 48 hours later and virus titers were determined on C6/36 by plaque assay and expressed as plaque forming unit per ml. Data are representative of two independent experiments. Data are shown as mean±SD.

FIG. 2. TYRO3 and AXL ectopic expression enhance DV Infection. Parental, TYRO3 and AXL-expressing 293T were infected with DV2 JAM (MOI of 10), DV2 NGC (MOI of 0.05), DV2 16681 (MOI of 0.05), DV1 TVP (MOI of 50), DV3 PAH881 (MOI of 5), DV4 1086 (MOI of 50). Percent of infected cells was quantified 48 hours later. Data are shown as mean±SD.

FIG. 3. TYRO3 and AXL ectopic expression enhance DV Infection. Parental, TYRO3 and AXL-expressing 293T were infected with DV2 JAM (MOI of 10), WNV (MOI of 0.0008), YFV-17D (MOI of 0.005), Influenza virus strain A/WSN/33 (1:5,000) or VSV pseudotyped HIV viral particle (100 ng p24). Infection was determined 48 hours later by FACS and normalized to infection in parental 293T cells. Data are shown as mean±SD.

FIG. 4. TYRO3 and AXL ectopic expression enhance DV Infection. Parental, TYRO3 and AXL-expressing HeLa cells were infected with DV3 (MOI of 30) and WNV (MOI of 0.001) and infection was scored 48 hours later by FACS using the 2H2 mAb or the anti-WNV E protein E16 mAb. Data are shown as mean±SD.

FIG. 5. DV and WNV infection of A549 is inhibited by downregulation of AXL. siRNA transfected A549 cells were infected with DV3 (MOI of 20) or WNV (MOI of 0.05). Percent of infected cells was quantified 24 hours later by FACS. Data are shown as mean±SD. *P<0.05, **P<0.01, ***P<0.001.

FIG. 6. DV and WNV infection of primary astrocytes is inhibited by downregulation of AXL. siRNA transfected primary astrocytes were infected with DV3 (MOI of 5) or WNV (MOI of 0.0001). Percent of infected cells was quantified 48 hours later. Data are shown as mean±SD. *P<0.05, **P<0.01, ***P<0.001.

FIG. 7. Polyclonal anti-human AXL Ab inhibits DV infection. 293T-AXL, A549 and primary astrocytes were incubated with either control goat IgG or goat anti-human AXL (10 μg/ml) antibodies 30 minutes before and throughout the 3 hours incubation with DV3 (MOI of 10). Infection level was quantified by FACS. Data are shown as mean±SD. *P<0.05, **P<0.01, ***P<0.001.

FIG. 8. Anti-human TYRO3 and anti-human AXL Ab inhibit an early step of DV infection. Parental, TYRO3 and AXL-expressing 293T were infected with DV2-JAM (MOI of 5). Indicated antibodies (10 μg/ml) were added 30 minutes prior and throughout infection (−30 min) or were added 2 hours post infection. Percent of infected cells was quantified 48 hours later by FACS. Data are shown as mean±SD. **P<0.01, ***P<0.001.

FIG. 9. TYRO3 and AXL enhance DV RNA uptake. Parental, TYRO3 and AXL-expressing 293T were incubated with DV2 JAM (MOI of 20) for 4 hours at 37° C. DV2 viral RNA level was determined by real-time quantitative PCR, using a comparative C_{T} method (ΔΔC_{T} method) with human GAPDH as endogenous control. Results are expressed as the fold difference using expression in 293T infected cells as calibrator value. The experiment was repeated two times with similar results. Data are shown as mean±SD. **P<0.01, ***P<0.001.

FIG. 10. TYRO3 and AXL mediate entry through a clathrin-dependent pathway. HeLa-TYRO3 and HeLa-AXL cells were reverse-transfected with indicated siRNA pool (20 nM) and infected with DV3 (MOI of 30) three days post-transfection. Percent of infected cells was quantified 48 hours later by FACS. Data are shown as mean±SD. **P<0.01, ***P<0.001.

FIG. 11. Soluble TYRO3 and AXL extracellular domains inhibit DV infection of 293T-TYRO3 and 293T-AXL. DV2 JAM (MOI of 10) was incubated with IgG1-Fc (control), TYRO3-Fc or AXL-Fc (10 μg/ml) 30 minutes and used for infection. Percent of infected cells was quantified 48 hours later by FACS. Data are shown as mean±SD. ***P<0.001.

FIG. 12. FBS component(s) enhances viral infection. 293T-TYRO3, 293T-AXL and 293T-DC-SIGN were infected with DV2-JAM (MOI of 10) in presence of different concentrations of FBS. After 3 hours incubation, medium was replaced with medium supplemented by 10% FBS. Percent of infected cells was quantified 48 hours later by FACS and normalized to the 10% infection condition. Data are shown as mean±SD. ***P<0.001.

FIG. 13. rmGas6 enhances DV binding to TYRO3 and AXL expressing cells. Parental, TYRO3 and AXL-expressing 293T were incubated with DV3 (MOI of 30) in serum free medium containing rmGas6 (1 μg/ml) or equivalent volume of PBS (mock) for 90 minutes at 4° C. Mean fluorescent intensity was measured by FACS and normalized to the MFI in non infected cells. Data are shown as mean±SD. ***P<0.001.

FIG. 14. rmGas6 enhance DV infection mediated by TYRO3 and AXL. 293T-TYRO3 and 293T-AXL were incubated with DV2 JAM (MOI of 5) in serum free medium containing rmGas6 (1 μg/ml) or equivalent volume of PBS (mock). After 3 hours incubation, medium was replaced by medium supplemented with 10% FBS. Percent of infected cells was quantified 48 hours later by FACS and normalized to infection in absence of rmGas6. Data are shown as mean±SD. ***P<0.001.

FIG. 15. rmGas6 interact with DV through its GIa domain. Coated DV2 JAM (10⁷ FIU) was incubated with rmGas6 or rmGas6ΔGIa (2 μg/ml) for 1 hour. Bound Gas6 was detected by ELISA using a goat polyclonal anti-Gas6 (10 μg/ml) and HRP-conjugated donkey anti-goat IgG. Data are shown as mean±SD. ***P<0.001.

FIG. 16. Annexin V inhibits TAM-mediated enhancement of DV infection. DV2 JAM (MOI of 5) was incubated with Annexin V (25 μg/ml) in serum free medium, and used to infect TYRO3, AXL and DC-SIGN-expressing cells. Percent of infected cells was quantified 48 hours later by FACS and normalized to infection in absence of Annexin V. Data are shown as mean±SD. ***P<0.001.

FIG. 17. Gas6ΔGIa does not bridge DV to TYRO3 and AXL. Coated DV2 JAM particle (10⁷ FIU) were incubated with indicated human Fc-chimeras (2 μg/ml) in presence or absence of rGas6 (2 μg/ml). Bound Fc-chimeras were detected using an HRP-conjugated rabbit anti-human IgG. Data are shown as mean±SD **P<0.01, ***P<0.001.

FIG. 18. Gas6ΔGIa inhibits DV infection enhancement mediated by TYRO3 and AXL. Cells were incubated with rmGas6 (10 μg/ml) or rmGas6 (1 μg/ml) 30 minutes before and throughout the 3 hours incubation with DV2 JAM (MOI of 5). Percent of infected cells was quantified 48 hours later. Data are shown as mean±SD **P<0.01, ***P<0.001.

FIG. 19. Gas6 ΔGIa inhibits DV-3 binding to TAM expressing CHO-745 cells. Data are shown as mean±SD **P<0.01, ***P<0.001.

FIG. 20. Schematic model of Gas6-mediated binding of DV and possible mechanisms of infection enhancement. The Gas6 function as bridging molecule by simultaneously binding to PtdSer exposed on the DV viral envelope, through the GIa domain, and to TAM receptor through the C-terminal SHGB-like domain. DV-Gas6 complexes could acts as “super” TAM receptors agonist and triggers a signal transduction cascade that results either in innate immunity inhibition or mobilization of endocytosis effectors that enhance virus internalization. Secondly, TAM receptors could act as an attachment factor, locally increasing the DV concentration and facilitating the interaction of the E protein with the virus bona fide receptor(s). Finally, DV-Gas6 may also induce recruitment of a virus bona fide receptor by heterotypic dimerization with TYRO3 or AXL, thereby forming a trimeric entry complex required for clathrin-mediated endocytosis of DV particles.

FIG. 21. TIM receptors mediate flavivirus infection. TIM receptors are used by DV2-JAM, West Nile Virus and Yellow Fever Virus. Parental and 293T cells expressing TIM receptors were infected by DV2-JAM, WNV (Israeli IS_{—}98-STI strain), Yellow Fever Virus vaccine strain (YFV-17D) and Herpes Simplex Virus 1 (HSV-1). Viral infection was quantified two days later by flow cytometry using specific Antibodies. Data are means±SEM of at least three independent experiments.

FIG. 22. TYRO3 and AXL enhance infection by DV and by other flaviviruses. Parental and TYRO3- and AXL-expressing 293T were challenged with DV2-Jam, WNV, YFV-17D and HSV-1. Infection was assessed 24 hours later by flow cytometry. Data are represented as mean±SEM from three independent experiments in duplicate.

FIG. 23. TIM-1 and TIM-4 ectopic expression enhance infection by Chikungunya. TIM1, TIM4 expressing 293T cells and parental 293T cells were infected with Chikungunya (Chick). Infection was quantified 48 hours later by flow cytometry, using a mouse monoclonal antibody against the E2 envelope glycoprotein (3E4).

FIG. 24. TYRO3 and AXL ectopic expression enhance infection by Chikungunya. TYRO, AXL expressing 293T cells and parental 293T cells were infected with Chikungunya (Chik). Infection was quantified 48 hours later by flow cytometry, using a mouse monoclonal antibody against the E2 envelope glycoprotein (3E4).

FIG. 25. Endogenous TIM-1 and AXL molecules mediate DV infection. A549 cells were infected with the indicated DV strains or HSV-1 in the presence of anti-TIM-1, anti-AXL or control IgG. The levels of infected were quantified 24h later by flow cytometry and normalized to infection in presence of control IgG. Data are means±SD of at least three independent experiments. **p<0.001, ***p<0.0001.

FIG. 26. Endogenous TIM-1 and AXL molecules mediate DV infection. Representative immunofluorescence analysis of A549 infected with DV2-JAM in the presence of the indicated Ab. Green anti-PrM 2H2, Blue DAPI. Scale bar: 100 μm. Data are means±SD of at least three independent experiments. **p<0.001, ***p<0.0001.

FIG. 27. Endogenous TIM-1 and AXL mediate DV infection. A549 cells were infected with DV3-PAH881 (MOI=10). Prior infection cells were incubated with indicated combination of anti-TIM-1 and anti-AXL polyclonal antibodies. Infection levels were quantified 24 hours later by flow cytometry and normalized to infection level in the presence of IgG control antibody. Means±SD from three independent experiments in duplicate are shown.

### EXAMPLES

### Material and Methods

cDNA Library Screening

For the cDNA screen, 1728 genes encoding putative cellular receptors were selected based on bioinformatics approach out of an arrayed full-length cDNA library (Porcel et al., 2004, Genome Res, 14:463-471). For primary round, 216 pools of 8 individual cDNAs (1 μg) were transiently transfected into 293T cells (24 well plate format) using Lipofectamine LTX (Life Technologies, Carlsbad, Calif.), according to the manufacturer's instructions. As positive control, equal amount of a DC-SIGN cDNA dilution (⅛^{th} in empty plasmid) was transfected. Empty plasmid was used as negative control. Transfection efficiency was assessed 24 hours post-transfection by immunostaining of ectopically expressed DC-SIGN. Next, transfected 293T cells were incubated with DV2 JAM primary strain (MOI of 2) for 48 hours and the percent of infected cell was measured by flow cytometry. In a second round, each positive cDNA pool and the 8 corresponding individual cDNAs (600 ng) were transfected into 293T cells and infected with DV2 JAM to identify individual positive cDNA.

### Elisa Binding Assay

96-well Maxisorp NUNC-IMMUNO plate (NUNC, Roskidle, Denmark) were coated overnight with DV viral particle (10⁷ FIU) at 4° C. in duplicate. Following blocking with 2% BSA in PBS CaCl₂/MgCl₂ at 37° C. for 1 hour, wells were incubated with rGas6 proteins (2 μg/ml) for 1 hour at 37° C. in TBS supplemented with 0.05% Tween and 10 mM CaCl₂. Wells were extensively washed, bound Gas6 proteins were labeled with Goat anti-Gas6 polyclonal Ab and detected with Horseradish peroxydase (HRP)-conjugated donkey anti-Goat IgG antibody (Santa Cruz Biotechnology, Heidelberg, Germany) (1:2,000 dilution) and o-phenylenediamine dihydrochloride (OPD) (Thermo scientific) substrate. For bridging experiments, rGas6 proteins (2 μg/ml) were simultaneously added during Fc-chimera proteins (2 μg/ml) incubation. Wells were extensively washed and bound Fc-chimeras were detected with Horseradish peroxydase (HRP)-conjugated rabbit anti-human IgG antibody (Dako) (1:1,000 dilution) and OPD.

### Cell Binding Assay

293T and CH0745 cells expressing TYRO3, AXL and DC-SIGN (4×10⁵) were incubated with indicated MOI of DV for 90 minutes at 4° C. in binding buffer (DMEM, NaN3 0.05%) containing 1% BSA or 5% FBS. For 293T, cells were incubated with 100 U of heparin for 30 min at room temperature before incubation with virus. The cells were washed two times with cold binding buffer, once with serum-free cold DMEM medium and fixed in PBS-PFA 2% at 4° C. for 20 minutes. Cell surface absorbed DV particles were stained with anti-panflavivirus envelope antibody (4G2, 5 μg/ml) and analyzed by flow cytometry as previously described (Fernandez-Garcia et al., 2011, J Virol, 85:2980-2989). In binding enhancement and inhibition assay, cells were incubated simultaneously with virus and rGas6 (10 μg/ml).

### Viruses and Cells

The DV-1-TVP strain, DV2-JAM strain (Jamaica), DV2-New Guinea C strain, DV2-16881 strain, DV3-PAH881 strain (Thailand) and DV4-1086 strain were propagated in mosquito (Aedes pseudoscutellaris) AP61 cell monolayers after having undergone limited cell passages. Of note, DV produced in mammalian cells gave similar results than viruses originating from insect cells. Virus titers were assessed by flow cytometry analysis (FACS) on C6/36 cells and were expressed as FACS infectious units (FIU). HEK 293T, A549, VERO, and Huh7 5.1 cells (a gift of C. Rice, New York, USA) were maintained in DMEM supplemented with 10% FBS, 1% penicillin/streptomycin.

DV2-JAM (Jamaica) and WNV (Israeli IS-98-STI strain was propagated in mosquito (Aedes pseudoscutellaris) AP61 cell monolayers as described above. YFV (strain YFV D17) was grown and titrated on Vero cells. HSV-1 (F) was propagated and titrated on Vero cells as described as described elsewhere (Taddeo et al. 2004). Chikungunya (strain CHIKV-21) was grown in insect cells C6/36.

### Flow Cytometry Analysis

Flow cytometry analysis was performed by following a conventional protocol in the presence of 0.02% NaN3 and 5% FBS in cold PBS. For infection assays, infected cells were fixed with PBS plus 2% (v/v) paraformaldehyde (PFA), permeabilized with 0.5% (w/v) saponin, followed by staining with mouse 2H2 mAb detecting DV prM (2 μg/ml), or mouse NS1 mAb detecting the nonstructural protein-1 (1 μg/ml). HSV-1 infection was detected with anti-ICP4 mouse mAb (clone 10F1, 0.3 μg/ml; Santa Cruz Biotechnology). WNV, YFV and Chikungunya infection were detected with the antibody anti-protein E (4G2) and a mouse monoclonal antibody against the E2 envelope glycoprotein (3E4). After 45 minutes, primary antibodies were labeled with a polyclonal goat anti-mouse immunoglobulin/RPE (DakoCytomation). Finally, infected cells percentages were assessed by flow cytometry on a LSR with CellQuest software (Becton Dickinson). Data were analyzed by using the FlowJo software (Tree Star).

### Statistical Analyses

Graphical representation, statistical analyses and curve fitting were performed using Prism5 software (GraphPad Software, San Diego, Calif.). Otherwise stated, all results are shown as means+/−standard deviation (SD) of 3 independent experiments. Results were tested for significance using paired two-tailed t test.

### Results

A cDNA Screen Identifies TYRO3 and AXL as Cell Surface Receptors that Enhance DV2 Infection

In an effort to identify new DV entry receptor(s), a gain of function cDNA screen for human genes that render poorly susceptible cell line 293T infectable by the DV2 strain JAM has been carried out. To this aim, sequence databases (Swiss Prot, Uniprot, Human Protein Reference Database) and selected 1728 full-length cDNAs encoding plasma membrane receptors from an arrayed cDNA library consisting of approximately 10000 cDNAs cloned into the CMV-driven expression vector pCMVSPORT6 were used. In the first round of screening, 216 pools of 8 cDNAs were transfected into 293T cells. It has been previously shown that 293T are poorly susceptible to infection by mosquito-derived DV primary strains, as only few cells infected were observed, even at a high multiplicity of infection (MOI). However, infectious particles from these cells transfected with the C-type lectin receptor DC-SIGN were efficiently recovered, indicating that restriction occurs at virus entry but not at viral replication, particle assembly or release steps. Transfected cells were then challenged with mosquito-grown DV2 JAM particles at a MOI of 2. Two day later, infection was scored by FAGS using the 2H2 mAb that recognizes the DV prM protein. Pools of cDNA that rendered 293T cells positive for prM protein intracellular staining entered the second round of screening, in which single cDNA composing each pool were individually tested. As a result for this screen, 3 main proteins were identified: L-SIGN, a C-type lectin receptor previously known to mediate DV entry, TYRO3 and AXL. TYRO3 and AXL are two members of the TAM (TYRO3/AXUMER) receptor family, a group of tyrosine kinase molecules that are activated upon binding of natural ligands, growth-arrest-specific 6 (Gash) and protein S (ProS). TAM receptors share an extracellular domain ligand binding domain (which includes two immunoglobulin like and two fibronectin type III repeats), a single-pass transmembrane domain and a cytoplasmic domain responsible for kinase activity. They are broadly expressed and regulate a variety of signaling cascades involved in cell transformation, phagocytosis, clearance of apoptotic cell bodies and innate immunity. Interestingly, one member of the TAM receptor family, AXL, has been found to facilitate Ebola virus and vaccinia virus infection.

### Ectopic Expression of TYRO3 and AXL Enhance DV1-4 Infection of Target Cells

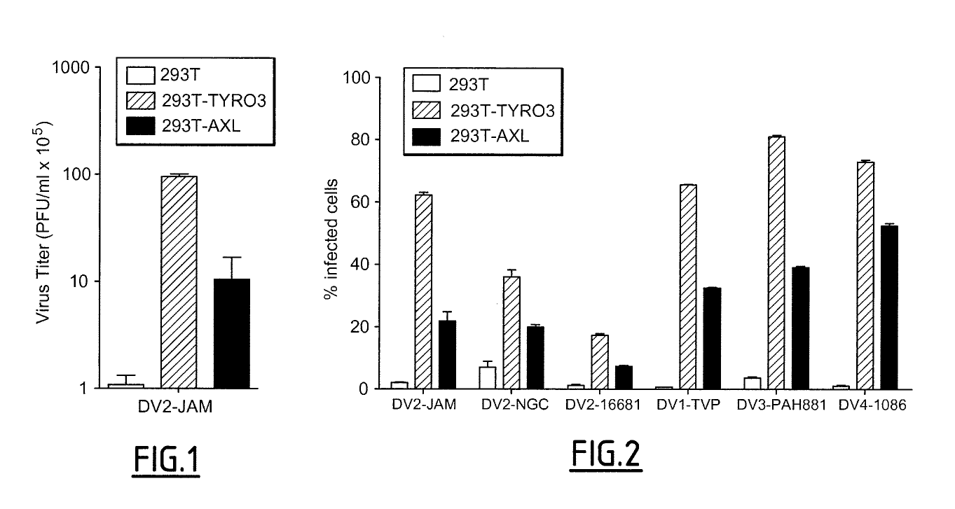

No enhancement of DV2 JAM infectivity with MER, the third member of the TAM receptor family was detected. The studies were thus focalized on TYRO3 and AXL. 293T cells, which express TYRO3 at a very low level and lack AXL expression, were next transduced with lentiviral vectors encoding human TYRO3 or AXL. Cells were stained with specific mAb and sorted for a high level of surface expression. Immunofluorescence studies showed that infection of 293T-TYRO3 or AXL cells with DV2 JAM resulted in a remarkable increase in prM protein intracellular production when compared to parental 293T cells. Consistently, FACS analysis of NS1 expression, a DV non-structural protein produced only during active replication, demonstrated that 293T-TYRO3 and 293T-AXL cells challenged with DV2 JAM are productively infected. Ectopic AXL or TYRO3 expression enhances DV2 infection of 293T cells by 20 and 50 fold respectively. Titration of cell-free supernatants collected from cells challenged with DV2 JAM showed that TYRO3 and AXL expressing cells released high amounts of infectious viral particles than the parental counterpart (FIG. 1).

Whether TYRO3 and AXL facilitate infection of human cells by any of the four DV serotypes was next tested. 293T-TYRO3, 293T-AXL and 293T cells were infected with a panel of DV1-4 strains. After 48h, cells were stained with the 2H2 mAb and analyzed by FAGS. Infection with the laboratory adapted strains DV2 NGC or 16881, as well as with the primary DV1, DV3 and DV4 strains was significantly enhanced by either TYRO3 or AXL (FIG. 2). Thus, ectopic expression of TYRO3 and AXL enhances infection of the four DV serotypes.

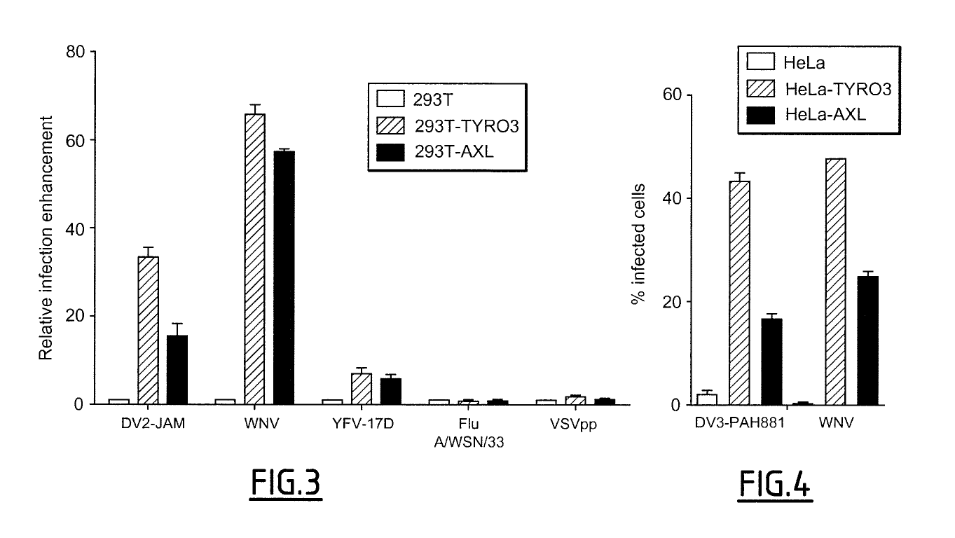

The specificity of TYRO3 and AXL-mediated enhancement of infectivity was next explored by testing other members of the flavivirus genus, WNV and YFV-17D, and other enveloped viruses such as Influenza (Flu) and HIV pseudoparticles bearing the vesicular stomatitis virus G protein (VSV pp). TYRO3 and AXL strongly enhanced WNV infection, and in a lesser extend YFV-17D. In contrast, no enhancement of viral infectivity occurred with Flu and VSVpp (FIG. 3). Thus, TYRO3 and AXL are exploited by different pathogenic flaviviruses for infection.

To determine whether TAM receptors expression renders other human cell lines susceptible to DV infection, HeLa cells stably expressing TYRO3 or AXL were generated. While parental HeLa cells are poorly sensitive to DV3 particles, efficient infection occurred after ectopic expression of TYRO3 or AXL (FIG. 4). Similar results were obtained with WNV (FIG. 4). Furthermore, the addition of TYRO3 in CHME cells, a macrophagic-like line that expresses endogenous AXL strongly enhance DV infection. Therefore, the addition of TYRO3 or AXL in at least three human cell lines enhances DV infection.

### Silencing or Inhibition of TAM Receptors Reduce DV Infection in Permissive Cells

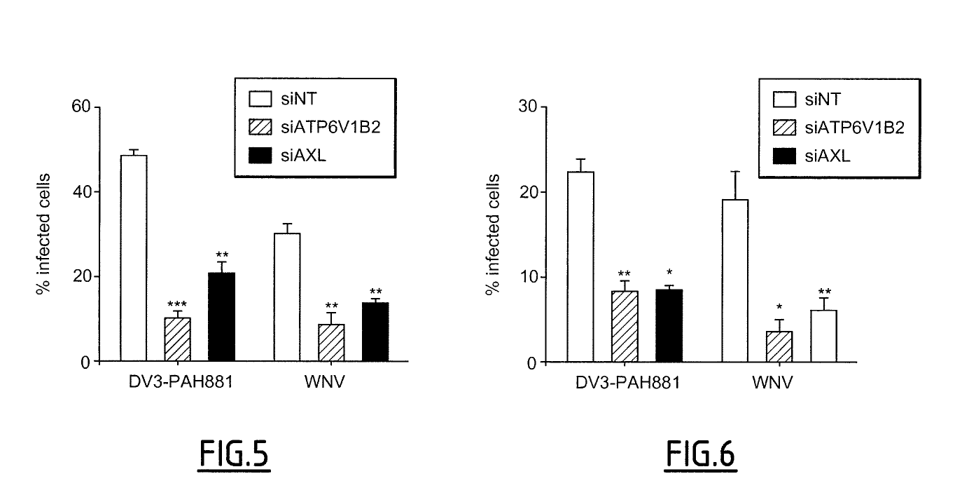

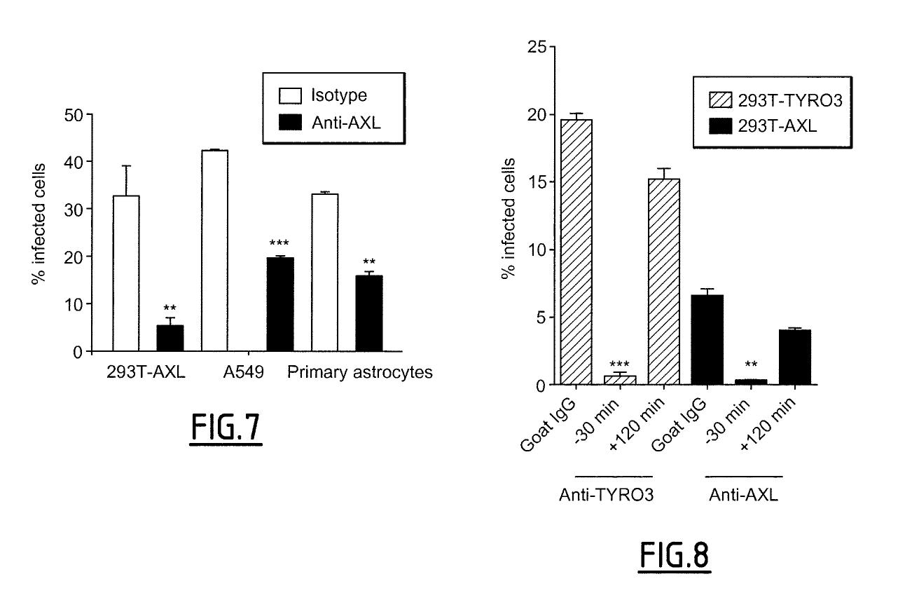

Flow cytometry experiments showed that AXL expression was detected in a wide range of human cells that are susceptible to DV infection. AXL is particularly abundant in A549 cells, as well as in primary human astrocytes, which have been proposed to be important DV targets in vivo. In contrast, TYRO3 was not detectably expressed in all the cells tested. Whether DV uses endogenous AXL in A549 cells and primary human astrocytes was then investigated. To this aim, expression of endogenous AXL was downregulated by siRNAs in both cell types as determined by flow cytometry. Susceptibility to DV3 and WNV infection was significantly decreased in AXL-silenced cells, compared with cells that received irrelevant siRNA. Silencing of ATP6V1 B2, a subunit of a vacuolar ATPase required for flavivirus pH-dependent fusion, impaired DV3 and WNV infection as efficiently as AXL siRNA (FIGS. 5 and 6). Similar results were obtained with DV2 JAM. The ability of antibodies directed against the AXL ectodomain to inhibit DV infection was then investigated. Pretreatment of 293T-AXL, A549 or primary astrocytes with anti-AXL Ab significantly inhibited DV3 and DV2 infection whereas control Ab had no effect (FIG. 7). Similarly, anti-TYRO3 Ab inhibited DV infection of 293T-TYRO3 cells (FIG. 8). Together, these results indicate that cell surface expression of AXL is required for optimal DV infection.

### TYRO3 and AXL Receptors Enhance DV Internalization

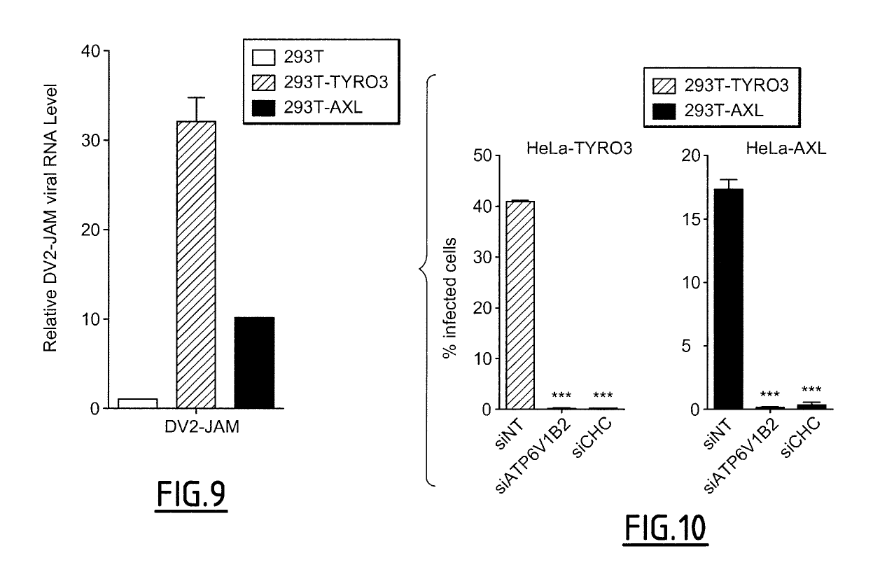

To determine at what step of the DV life cycle TYRO3 and AXL act, the ability of anti-TAM Ab to inhibit DV infection, when added at various time point was first examined (FIG. 8). Anti-TYRO3 and anti-AXL Ab strongly inhibited DV infection when respectively incubated with 293T-TYRO3 or 293T-AXL 30 min prior virus challenge (FIG. 8). The Abs lost their neutralizing ability when added 2h post-infection (FIG. 8), suggesting that TAM receptors act at an early step of the DV life cycle. Whether TYRO3 and AXL promote DV internalization was then investigated. TYRO3 and AXL expressing cells were challenged with DV2 JAM for 4h at 37° C. Total RNA was extracted and viral RNA levels were quantified by qPCR (FIG. 9). TAM receptors strongly increased DV RNA uptake into 293T cells (30 fold and 10 fold enhancement with TYRO3 and AXL, respectively) (FIG. 9). In a second approach, TYRO3 and AXL were expressed into CHO-745 cells, which lack cell surface heparan sulfate. TAM-expressing cells or, as a control, DC-SIGN CHO-745 cells were incubated with DV2 JAM particles for 1h at 4° C. and shifted at 37° C. for 45 min to allow endocytosis. Virus uptake was monitored by fluorescence microscopy using anti-DV E mAb 4G2. As with DC-SIGN, we found a strong intracellular accumulation of DV E protein in cells expressing TYRO3 or AXL. Therefore, DV particles are efficiently internalized into target cells upon TYRO3 or AXL expression. These results indicate that the TAM receptors TYRO3 and AXL are novel cell entry cofactors for DV.

Whether DV entry in TAM-expressing cells requires a functional clathrin-dependent endocytic pathway was then investigated. HeLa cells expressing TYRO3 or AXL were transfected with siRNA targeting the clathrin heavy chain (CHC), a cellular factor that promotes coated pit formation. Silencing was verified by WB and by a functional assay demonstrating inhibition of clathrin-mediated uptake of transferin as previously described. CHC silencing potently inhibited DV infection of TYRO3 and AXL-expressing cells (FIG. 10). As a positive control, ATP6V1 B2 silencing also blocked DV infection. Together, these data showed that TAM-mediated enhancement of DV entry is pH-dependent and involves the clathrin pathway.

Soluble Gas6 Interacts with PdtSer Expressed on DV Envelope and Bridges Viral Particles to TYRO3 and AXL

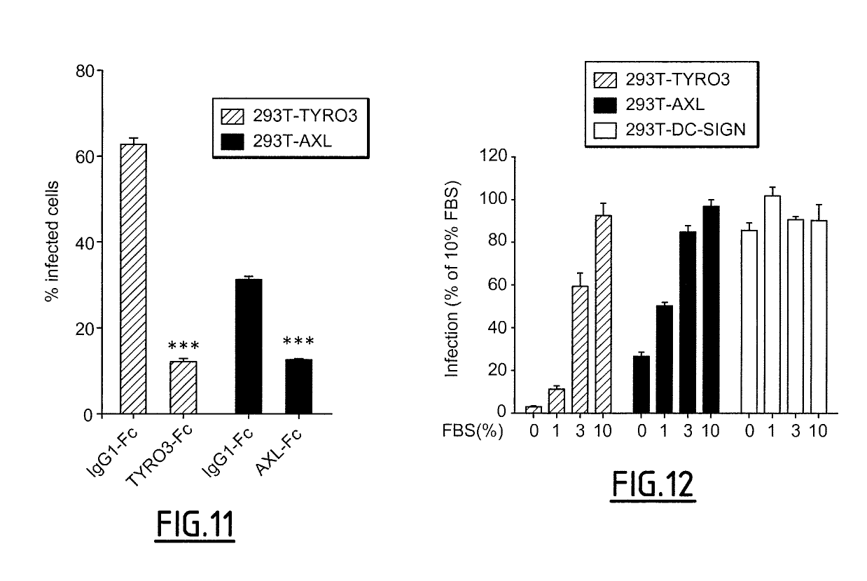

To elucidate the mechanisms by which TYRO3 and AXL enhance DV entry, an inhibition infection assay was performed with soluble chimeric TYRO3-Fc and AXL-Fc molecules. Parental 293T-TYRO3 or 293T-AXL cells were infected with DV2 JAM particles preincubated with TYRO3-Fc, AXL-Fc or control Fc molecules (FIG. 11). DV infection was significantly blocked by soluble TYRO3-Fc or AXL-Fc, strongly suggesting that DV virions bind to TAM receptors. However, pull-down experiments using soluble TYRO3-Fc or AXL-Fc failed to immunoprecipitate DV-E protein from intact DV particles, indicating that TYRO3 or AXL ectodomain do not directly interact with DV. To further investigate how TAM receptors associate with DV, virus attachment assays was conducted in the presence or absence of fetal bovine serum (FBS) which contains high levels (−300nM) of the TAM ligand ProS. Parental, TYRO3, AXL 293T cells were incubated at 4° C. with DV particles with or without 5% FBS. DV particles bound to the cell surface were detected by FACS using the anti-E protein 4G2 mAb. A significant increase in virus binding to TYRO3 and AXL was detected only in the presence of FBS. Similar results were obtained when TYRO3 or AXL were expressed on CHO 745 cells and with various DV strains. With FBS, binding of DV to TAM-positive cells was specific since it was inhibited by anti-TYRO3 or anti-AXL Ab. Importantly the TAM effect on DV was abrogated when infections were performed in the absence of FBS (FIG. 12). Increasing the FBS concentration to 10% enhanced DV infection of TYRO3 or AXL but not DC-SIGN expressing cells (FIG. 12). Collectively, these data indicate FBS factors may facilitate DV binding to TAM receptors, allowing enhanced virus entry.

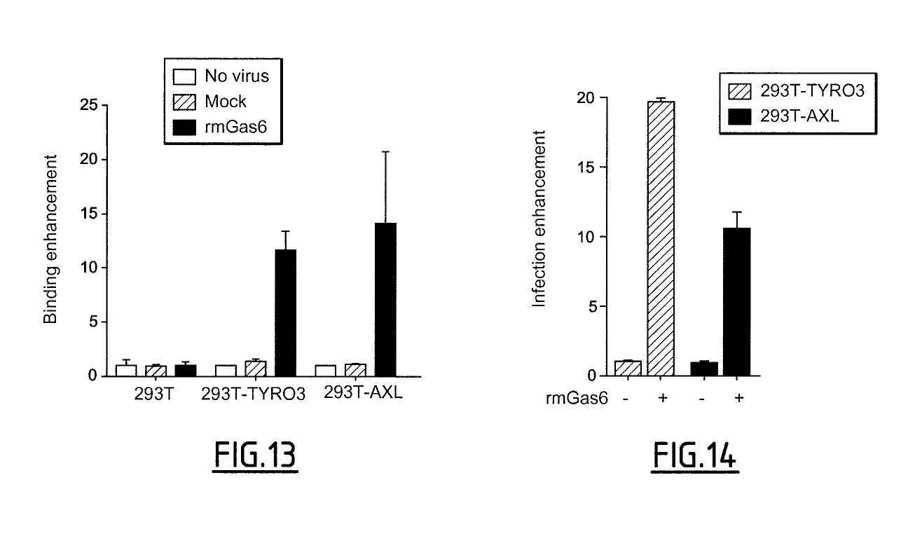

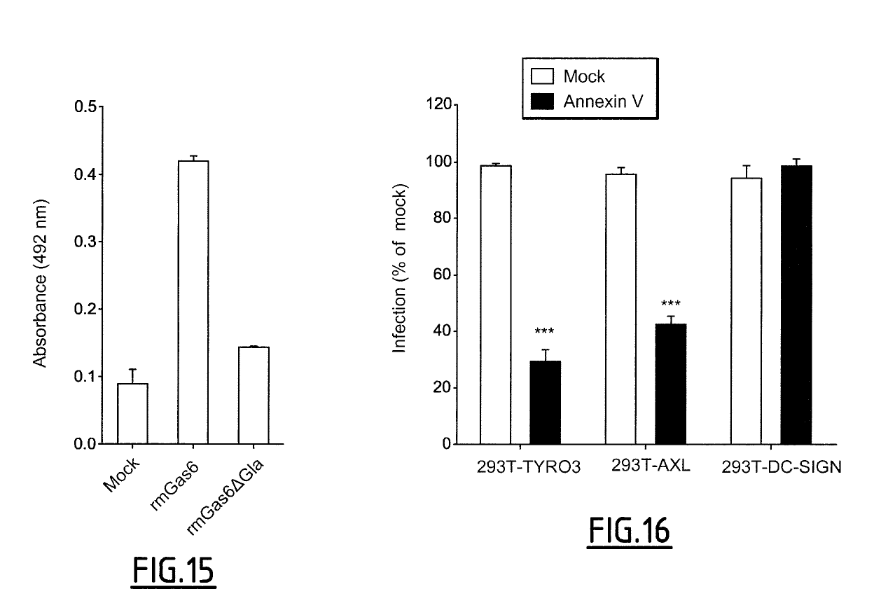

Whether DV binding to TAM receptors is modulated by full-length murine Gas6 (rmGas6) was investigated. DV binding to TYRO3 and AXL expressing cells was significantly increased when viral particles were preincubated with mGas6 but not with the mock control (FIG. 13). Consistently, full-length rmGas6 drastically boosted DV infection of TYRO3 and AXL expressing cells and not of control cells (FIG. 14). Similar results where obtained using human full length Gas-6. This strongly suggested that Gas6 complexed to DV interacts with TAM receptors to enhance DV entry. In order to know if Gas6 recognizes PdtSer expressed on DV virions, an ELISA assay where DV coated on wells were incubated with various Gas6 molecules was performed (FIG. 15). Full-length Gas6 efficiently binds to DV particles. In contrast, a Gas6 molecule lacking the N-terminal GIa domain (rmGas6ΔGIa) and thus unable to bind PdtSer was impaired in its ability to interact with DV particles.

It has then investigated if PdtSer expressed on DV particles are required for infection of TAM-positive cells. TYRO3 and AXL cells were infected with DV preincubated with annexin V (ANX5), a well-documented PdtSer binding protein. ANX5 has no effect on DV entry through DC-SIGN. In contrast, ANX5, probably by blocking access to PdtSer, significantly inhibited DV infection of 293T cells expressing TYRO3 or AXL and cultured in FBS (FIG. 16). Altogether, the results showed that Gas6 enhanced DV entry. The molecule acts by bridging PdtSer exposed on DV virions to the TAM receptors TYRO3 and AXL.

### Antiviral Activity of Gas6 Molecule Unable to Bridge DV Particles to TYRO3 and AXL

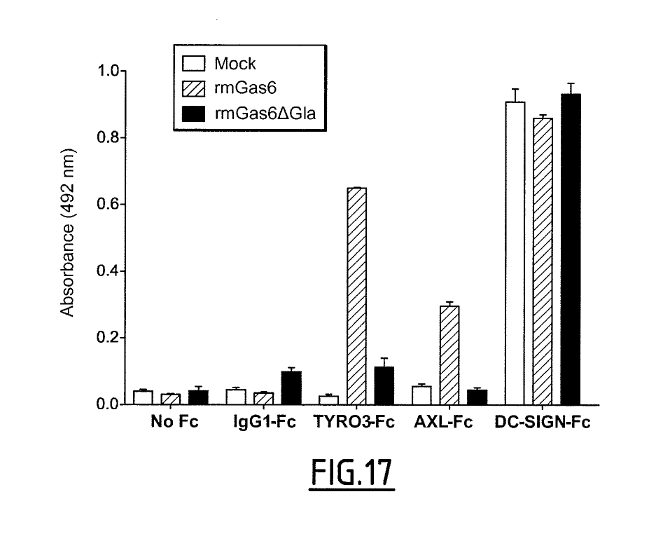

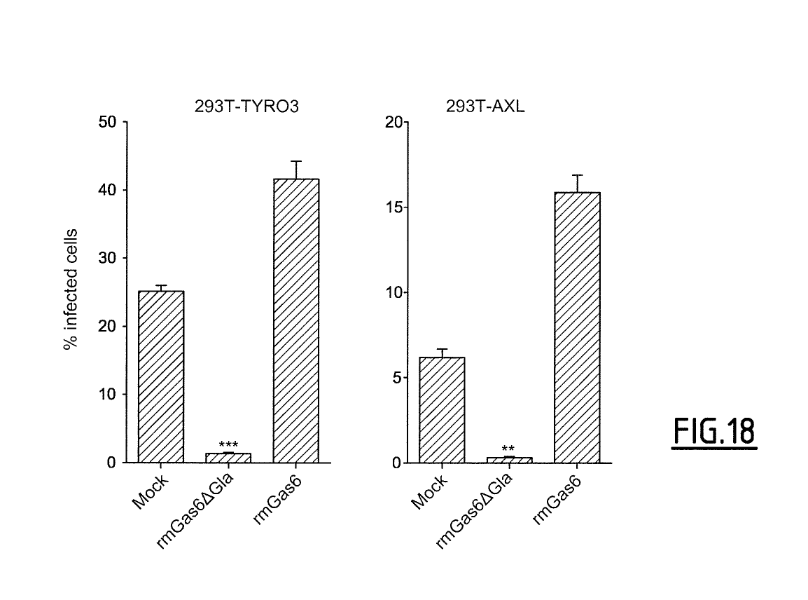

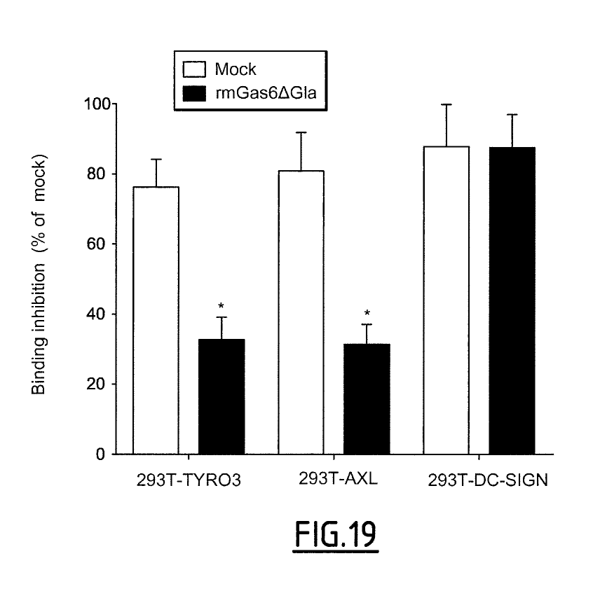

The capacity of full-length mGas6 and a commercially available Gas6 derivative lacking the GIa domain (rmGas6ΔGIa) to modulate DV binding and infection of TYRO3 and AXL-expressing cells was next compared. The two Gas6 proteins were tested for their ability to bridge DV to TYRO3 and AXL. DV particles coated on ELISA plates were incubated with soluble TYRO3-Fc, AXL-Fc or as control irrelevant Fc molecules in the presence of the Gas6 molecules. In this assay, the full-length rmGas6, displayed DV bridging activity whereas this was not the case for rmGas6ΔGIa and surprisingly rhGas6 (FIG. 17). As control, none of the Gas6 molecules tested modulated binding of DV to DC-SIGN-Fc. It was observed that full-length rmGas6 enhanced DV infection even in the presence of FBS (FIG. 18). This is consistent with the finding of very low serum concentrations of Gas6 (0.2-0.5 nM), almost all of which is complexed with soluble Axl ectodomain. Interestingly, pretreatment of TYRO3 or AXL-expressing 293T cells with rmGas6ΔGIa abrogated enhancement of DV2 infection (FIG. 18). These binding experiments indicated that inhibition of TAM-receptor mediated infection by rmGas6ΔGIa correlated with the ability of this molecule to block DV3 binding to TAM-expressing CH0745 cells (FIG. 19). Altogether, these results show that a Gas6 molecule unable to attach DV to TAM receptors functions as an antiviral compound.

### TIM and TAM Receptors Mediate Infection by Other Flavivirus

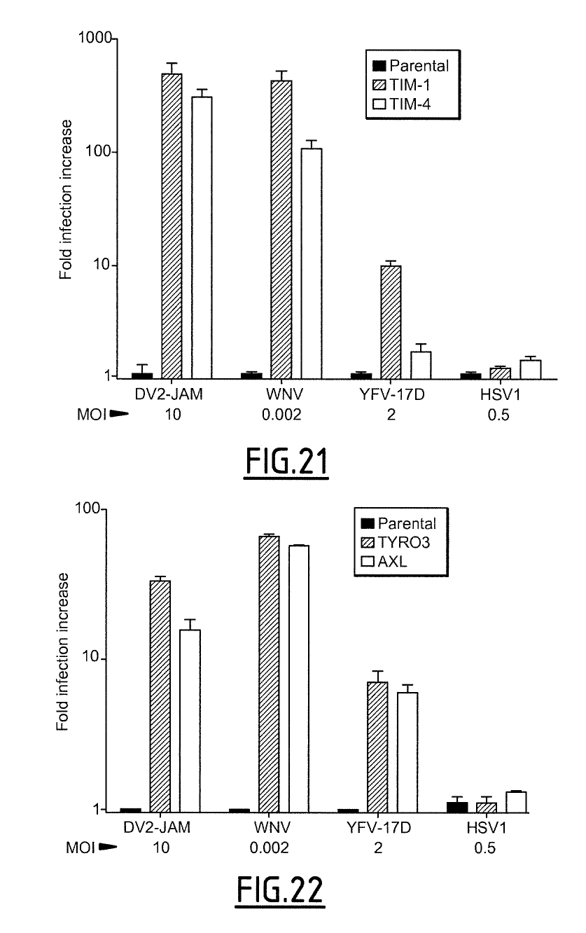

To determine whether TIM and TAM receptors mediate infection by other viral species, TIM-1- and TIM-4-expressing cells were challenged with DV2-Jam West Nile virus (WNV), Yellow Fever Virus vaccine strain (YFV-17D), and Herpes Simplex Virus 1 (HSV-1). Viral infection was quantified by flow cytometry using specific Antibodies (FIG. 21). The data show that TIM-1 and TIM-4 massively enhanced WNV infection, slightly upregulated sensitivity to YFV-17D, but had no effect on HSV-1. Similar results were obtained for TYRO3- and AXL-expressing cells (FIG. 22). Together, these data indicate the PtdSer receptors TIM and TAM are both cellular factors promoting flavivirus infection.

### TIM and TAM Ectopic Expression Enhance Infection by Chikungunya

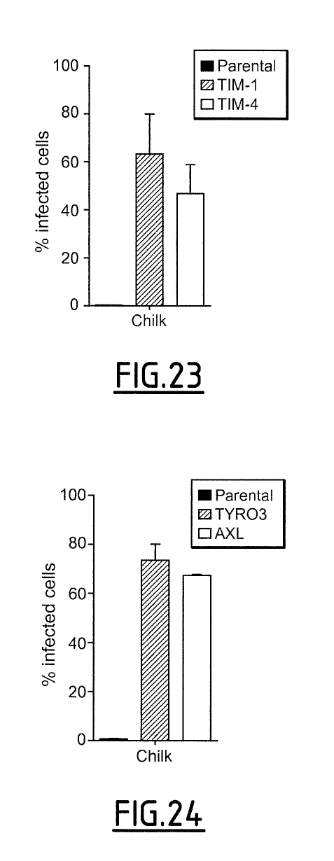

Furthermore, it was of interest if this mechanism represents a general mechanism exploited by viruses that express or incorporate PtdSer in their membrane. Parental 293T cells, TIM-1 and TIM-4 expressing 293T cells were infected with Chikungunya (Chick). Infection was quantified 48 hours later by flow cytometry using a mouse monoclonal antibody against the E2 envelope glycoprotein (3E4). The results (FIG. 23) show that TIM-1 and TIM-4 massively enhance Chikungunya infection. Similar results were obtained for TYRO3 and AXL expressing cells, their ectopic expression enhances as well Chikungunya infection (FIG. 24).

These data show that TIM and TAM facilitation of viral infection represents a general mechanism exploited by viruses that express or incorporate PtdSer in their membrane for optimal infection.

### Endogenous TIM-1 and AXL Molecules Mediate DV Infection

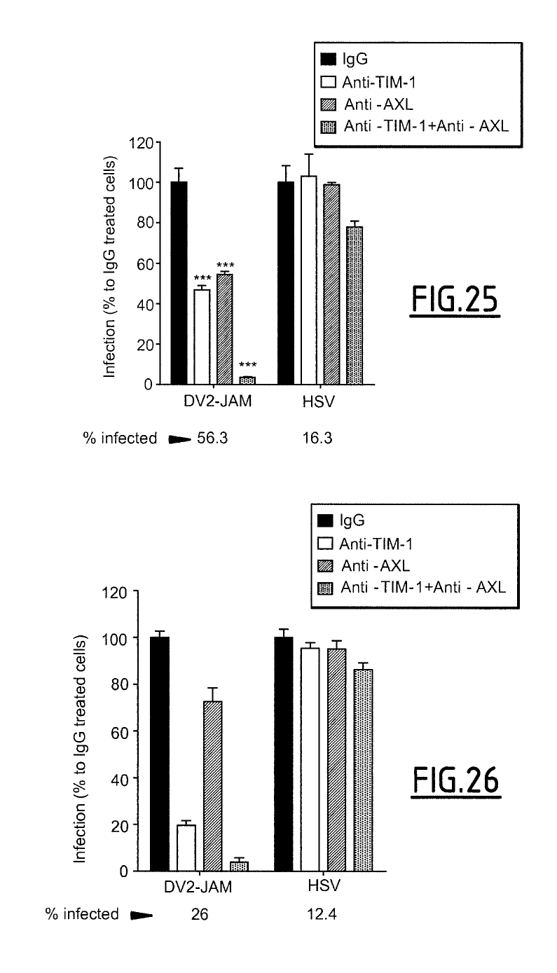

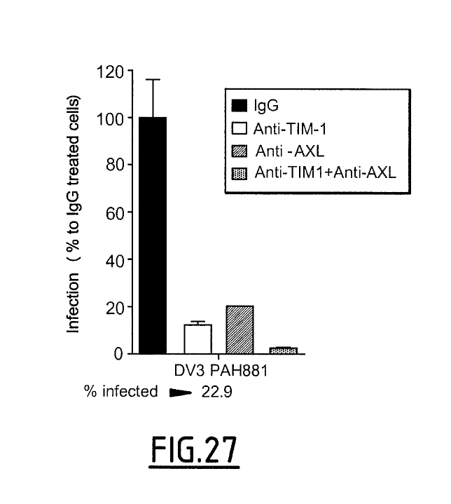

The A549 cell line expresses both TIM-1 and AXL. DV2 infection was partly reduced with an anti-TIM-1 or anti-AXL Ab administrated alone, while the two Ab in combination fully inhibited DV2 (FIGS. 25 and 26), DV3 (FIG. 27) but not HSV-1 infection. Similar results were obtained in Vero cells that express TIM-1 and AXL. These results show that TIM and TAM receptors may naturally cooperate to promote DV infection and that PtdSer is mediating infection in cells endogenously expressing the receptors.

### Discussion

The current study adds significant insights into the molecular mechanisms and cellular requirements for DV entry. Using a gain-of-function cDNA screen, the inventors identified TYRO3 and AXL as novel DV entry cofactors. TYRO and AXL constitute with MER the TAM family of receptor tyrosine kinases (RTKs) which regulates an intriguing mix of processes and are essential for the phagocytosis of apoptotic cells. TYRO3 and AXL recognize PdtSer exposed on the viral envelope through their natural ligand Gas6 and ProS which bridge DV virions to the host cell and enhance virus internalization. These finding thus indicate that DV manipulate host receptors involved in the clearance of apoptotic cell bodies for its infectious entry and suggest that DV exploit multiple cell receptor system to ensure that it can establish a productive infection in the human host.

This study demonstrated that DV particle did not directly interact with TAM receptors. On the contrary, enhancement of DV infection and absorption on TYRO3 and AXL-expressing cells was almost entirely dependent on the presence of serum. Serum contains two TAM receptor ligands, the vitamin K-dependent protein Gas6 and the closely related anticoagulant protein S that are present in the plasma at physiological concentration of 0.25 nM and 350 nM respectively. Both molecules are responsible for bridging apoptotic cells to TAM receptor and Gas6 was recently reported to enhance lentiviral pseudotyped virus transduction mediated by AXL. Interestingly, similar to these results in presence of FBS, Gas6 enhanced DV infection and absorption on TAM expressing cells in absence of serum. On the contrary, Gas6 lacking the GIa domain decreased infection and TAM receptor binding in presence of serum. These experiments suggest that the component present in the serum use a similar mechanism and bind to a common or overlapping TAM receptor domain than Gas6. Thus, it is likely that Gas6 and/or ProS present in the serum when attached to the virus through its GIa domain, binds simultaneously to the TAM receptor through its SHBG domain. Inventors's observation brought new evidences that flavivirus interaction with cellular receptor could involve a bridging molecule and strengthened the recent study on WNV indirect binding to mosquito cellular receptor mosPTP-1. In the case of WNV, the soluble C-type lectin mosGCTL-1 bridged WNV envelope protein to mosPTP-1 and subsequently facilitated infection.

Several evidences suggest that the FBS effects observed are certainly due to ProS rather than Gas6. First the concentration of Gas6 in plasma is very low and nearly all Gas6 is complexed with soluble AXL ectodomain (sAXL). On the contrary, ProS concentration is very high and only 60% of ProS is complexed with C4BP (C4b-binding protein). Secondly, Gas6 binds to all three TAM receptors in vitro (AXL≧TYRO3>>MER) and indeed Gas6 similarly enhanced DV binding to TYRO3 and AXL-expressing cells as observed in FIGS. 11-16. In contrast, ProS is a more potent in vitro ligand for TYRO3 than AXL, which is consistent with the strong FBS enhancement of DV binding to TYRO3-expressing cells and to AXL-expressing cells in a lesser extend.

Since Gas6 and protein S bridging activity suggested an interaction of the GIa domain with the viral particle, the inventors hypothesized that PtdSer residues are exposed on DV viral envelope. The ELISA experiments described herein revealed indeed that the GIa domain of Gas6 bound the virus and that the potentiating effect of serum is competed away by prior incubation of the viral particle with the PtdSer-binding protein Annexin V. Interestingly, PtdSer seems to be exposed on several enveloped virions and previous publications have shown that Annexin V binds to HIV-1, vaccinia virus, CMV, HSV-1 and HSV-2. In agreement with the DV life cycle, it is assumed that DV acquired PtdSer while it budded in the lumen of the endoplasmic reticulum (ER). The plasma membrane and the ER bilayer membrane are composed of two leaflets and in resting cell PtdSer is localized and enriched in their inner leaflet. While budding from the cytosol, the nucleocapsid induced an inward invagination of the ER membrane. During this process, the inner leaflet of the ER membrane and the prM-E heterodimers, which were anchored on the luminal side of the ER, are exposed at the surface of the enveloped viral immature particle.

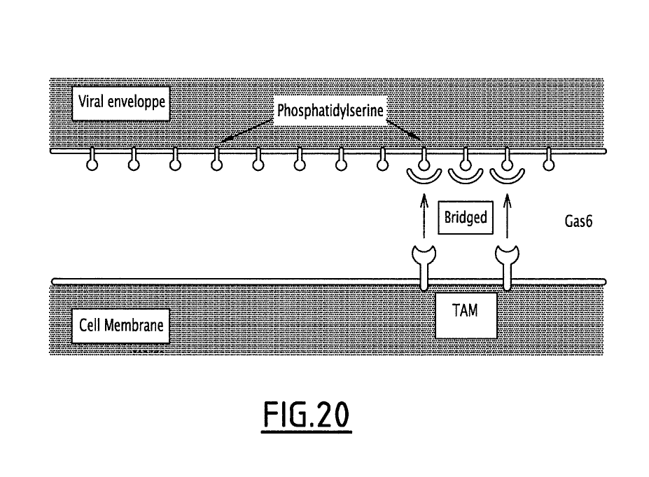

The involvement of TYRO3 and AXL in DV entry raises interesting questions about how, upon virus-ligand complex binding, these molecules facilitate infection. As schematized in FIG. 20, TYRO3 and AXL proteins could facilitate the interaction of the viral envelope protein with its primary receptor, for example by increasing the cell surface virus concentration, in a model similar to DC-SIGN-mediated DV entry. Furthermore, because TAM receptors can physically associates with non-TAM receptor by heterotypic dimerization, it is conceivable that TYRO3 and AXL can recruit the bona fide receptor. This interaction may lead to activation of downstream signal pathway enhancing clathrin-mediated DV internalization. Another appealing hypothesis is that the DV-TAM ligand complex binding could triggers receptor activation of downstream effectors facilitating viral infection. This hypothesis is supported by: (i) Gas6 binding to TAM receptor triggers a signal transduction cascade with a variety of cell-dependent biological outcomes; (ii) Demonstration that cytoplasmic tail deletion, as well as mutation of an ATP binding site (K567M) that abolished tyrosine phosphorylation reduced AXL-mediated enhancement of ZEBOV-GP transduction; (iii) Phospholipase C pathway activation is required for AXL-dependent cell transduction by virus pseudotyped with ZEBOV-GP. Therefore it is conceivable that downstream pathways activation is required for TYRO3/AXL-mediated DV infection enhancement.

Actually, one of these downstream pathways could be the negative regulation of innate immunity. Recently it has been reported that Gas6 activated TAM receptor function as an inhibitor of the inflammation induces by Toll-like receptor (TLR) and cytokine receptor in macrophage and dendritic cells. During inflammation, TLR and cytokine signaling drove upregulation of AXL expression, which subverted the pro-inflammatory INFAR/STAT1 signaling pathway to induce transcription of SOCS1 and SOCS3 (suppressor of cytokine signaling) gene and negatively regulated innate immunity and inflammation. Gas6 binding to AXL induced a 10 fold increase of SOCS1 mRNA expression and inhibited TLR-induced cytokine production. One can assume that DV-TAM ligand complex could act as a superagonist of TAM receptor and induced a potent receptor activation that stimulates SOCS gene expression and subsequent TLR inhibition, thus facilitating the early stage of infection.

A model whereby DV through PtdSer residues present on the viral envelope is recognized by a serum component as an apoptotic body, thereby forming a complex that has the ability to bind to cell surface TYRO3 and AXL is predicted. By mimicking apoptotic bodies, DV subverts the apoptotic clearance function of TAM receptor to facilitate infection.

1. a method for preventing or treating a viral infection comprising administering to an individual in need thereof a therapeutically effective amount of an inhibitor of an interaction between phosphatidylserine and a TAM receptor, wherein said inhibitor is:
(i) a TAM receptor inhibitor,
(ii) a Gas6 inhibitor, and/or
(iii) a phosphatidylserine binding protein.

2. A method according to claim 1 (i), wherein said TAM receptor is TYRO3, AXL or MER.

3. A method according to claim 1 (i), wherein said TAM receptor inhibitor is an anti-TAM receptor antibody, an antisense nucleic acid, a mimetic or a variant TAM receptor.

4. A method according to claim 1 (ii), wherein said Gas6 inhibitor is an anti-Gas6 antibody, an antisense nucleic acid, a mimetic or a variant Gas6 protein.

5. A method according to claim 1 (iii), wherein said phosphatidylserine binding protein is an anti-phosphatidylserine antibody or Annexin 5.

6. A method according to claim 3, wherein said TAM receptor inhibitor is a siRNA of sequence SEQ ID NO: 1, 2, 3, or 4.

7. A method according to claim 4, wherein said Gas6 inhibitor is the variant Gas6 protein Gas6AGIa of sequence SEQ ID NO: 19.

8. A method according to claim 1, wherein said virus is a phosphatidylserine harboring virus.

9. A method according to claim 1, wherein said phosphatidylserine harboring virus is an Alphavirus or a Flavivirus.

10. A method according to claim 9, wherein said Alphavirus virus is Chikungunya virus.

11. A method according to claim 9, wherein said Flavivirus is a West-Nile Virus, Yellow Fever Virus or Dengue Fever Virus.

12. A method according to claim 1, wherein said inhibitor is for administration in combination with at least one other antiviral compound, either sequentially or simultaneously.

13. A method according to claim 9, wherein said other antiviral compound is an inhibitor of an interaction of phosphatidylserine and a TIM receptor.

14. A method according to claim 1, wherein said inhibitor is formulated in a pharmaceutically acceptable composition.

15. A pharmaceutical composition comprising an inhibitor as defined in claim 1 and additionally at least one other antiviral compound.

16. A pharmaceutical composition according to claim 15, wherein said at least one antiviral compound is an inhibitor of an interaction of phosphatidylserine and a TIM receptor.

17. A method of inhibiting entry of a phosphatidylserine harboring virus into a cell comprising exposing said cell to an inhibitor as defined in claim 1.

18. (canceled)
